(19) United States
(12) Patent Application Publication
(10) Pub. No.: US 2025/0002918 A1
(43) Pub. Date: Jan. 2, 2025

(54) COMPOSITIONS AND METHODS FOR TREATMENT OF MIC60 DEPLETED CANCERS AND METASTASIS

(71) Applicant: The Wistar Institute of Anatomy and Biology, Philadelphia, PA (US)

(72) Inventor: Dario C. Altieri, Philadelphia, PA (US)

(21) Appl. No.: 18/703,004

(22) PCT Filed: Oct. 22, 2022
(86) PCT No.: PCT/US2022/078567

§ 371 Date: Apr. 19, 2024

Related U.S. Application Data
(60) Provisional application No. 63/371,600, filed on Aug. 16, 2022.
(60) Provisional application No. 63/270,883, filed on Oct. 22, 2021.

(51) Int. Cl. C12N 15/113; A61K 31/12; A61K 31/17; A61K 31/282; A61K 31/36; A61K 31/437; A61K 31/4427; A61K 31/454; A61K 31/4725; A61K 31/4745; A61K 31/506; A61K 31/513; A61K 31/519; A61K 31/5377; A61K 31/635; A61K 45/06; A61P 35/00; G01N 33/574; G01N 33/68

(52) U.S. Cl. CPC C12N 15/1137; A61K 31/12; A61K 31/17; A61K 31/282; A61K 31/36; A61K 31/437; A61K 31/4427; A61K 31/454; A61K 31/4725; A61K 31/4745; A61K 31/506; A61K 31/513; A61K 31/519; A61K 31/5377; A61K 31/635; A61K 45/06; A61P 35/00; G01N 33/57496; G01N 33/6893; C12N 2310/14; C12N 2310/531; G01N 2474/20

(57) ABSTRACT

Provided herein are methods of treating cancer in a subject in need thereof. The method includes obtaining a tumor sample from the subject and detecting an expression level(s) of one or more genes in a tumor sample. In certain embodiments, the genes comprise MMP13, XAF1, IGFBP3, MX2, IFIT1, ISG15, OAS3, CXCL10, SERPINE1, CXCL11, and CXCL3. A change in the expression level of the one or more genes as compared to a control level indicates Mic60-depleted cancer. The method further includes treating the subject for Mic60-depleted cancer.

### STATEMENT REGARDING FEDERALLY SPONSORED RESEARCH OR DEVELOPMENT

This invention was made with government support under grant numbers CA140043, CA220446, CA221838, CA211199, and OD023586 awarded by the National Institutes of Health. The government has certain rights in the invention.

### BACKGROUND

The rewiring of metabolic pathways is a ubiquitous cancer trait that confers cellular plasticity, expands clonal heterogeneity, and enables disease progression. There is now a consensus that mitochondria are important for this process, titrating energy output, buffering oxidative stress, and controlling a host of cell death programs. In particular, exploitation of mitochondrial functions has been linked to metastatic competence. This involves oxidative bioenergetics and redox balance but also deregulated mitochondrial dynamics, a process that controls the size, shape, and distribution of mitochondria and their trafficking to the cortical cytoskeleton, where they fuel pivotal steps of cell motility, such as membrane lamellipodia dynamics, turnover of focal adhesion (FA) and phosphorylation of signaling kinases.

However, the environment of tumor growth is highly unfavorable to mitochondrial fitness. Erratic oxygen concentrations, high levels of oxidative radicals, constantly changing metabolic needs, and vulnerabilities of the mitochondrial proteome are all potent stimuli to disrupt mitochondrial integrity, shut off organelle functions, and activate cell death. Quality-control measures activated in these settings, in particular mitophagy, are designed to remove such subpar, “ghost” mitochondria and restore homeostasis. However, the role of these pathways in cancer is far from clear, and activation of mitophagy has been paradoxically linked to tumor progression as well as treatment resistance.

An important regulator of mitochondrial integrity is Mic60, also called Mitofilin or inner membrane mitochondrial protein. Mic60 is an essential constituent of a MICOS complex that maintains cristae architecture, organizes respiratory complexes, and ensures outer membrane biogenesis. Whether this pathway is important in cancer has not been determined, but evidence exists that Mic60 participates in mitochondrial fitness, including PINK1/Parkin-directed mitophagy and mitochondrial dynamics.

What is needed is improved compositions and methods for treatment of cancer.

### SUMMARY OF THE INVENTION

In one aspect, provided herein is a method of treating cancer in a subject in need thereof, the method comprising: a) obtaining a tumor sample from the subject; b) detecting expression levels of genes in the tumor sample, wherein the genes comprise MMP13, XAF1, IGFBP3, MX2, IFIT1, ISG15, OAS3, CXCL10, SERPINE1, CXCL11, and CXCL3, wherein the expression level(s) of the one or more genes as compared to a control level indicates Mic60-depleted cancer; and c) treating the subject for the Mic60-depleted cancer.

In one aspect, provided herein is a method of treatment for cancer in a subject in need thereof, the method comprising: having obtained information about expression levels of genes in a tumor sample obtained from the subject, wherein the genes comprise MMP13, XAF1, IGFBP3, MX2, IFIT1, ISG15, OAS3, CXCL10, SERPINE1, CXCL11, and CXCL3, wherein the expression level(s) of one more of the genes as compared to a control level indicates Mic60-depleted cancer, administering a treatment for the cancer to the subject.

In another aspect, provided herein is a method of reducing, inhibiting, and/or preventing tumor metastasis in a subject in need thereof, the method comprising: having obtained information about expression levels of genes in a tumor sample obtained from the subject, wherein the genes comprise MMP13, XAF1, IGFBP3, MX2, IFIT1, ISG15, OAS3, CXCL10, SERPINE1, CXCL11, CXCL3, wherein a the expression level(s) of the one or more genes as compared to a control level indicates Mic60-depleted cancer, administering a treatment for the cancer to the subject.

In certain embodiments, the genes further comprise one or more of IFIT3, IFITM1, IFIT1, OAS1, IFI35, MX1, ISG15, IFI6, IFITM2, TAP1, STAT1, IFITM3, STAT2, PSMB8, IRF1, IFNB1, IRF9, IFNGR1, RELA, BAK1, XAF1, IGFBP3, MX2, IFIT1, IFIT2, CXCL10, SERPIN1, OAS2, MX1, ISG15, OAS3, IL6, IL8, MMP13, CXCL3, CXCL2, CCL26, CCL20, VEGFC, IL15, CXCL6, CXCL1, SERPRINB2, ATF5, CD55, KITLG, IGFBP6, CD276, LAMB2, ANG, ETS2, INHBA, AXL, AREG, RELA, and MIF. In other embodiments, the genes consist of XAF1, IGFBP3, MX2, IFIT1, ISG15, OAS3, CXCL10, SERPINE1, CXCL11, CXCL3, and MMP13.

In certain embodiments, the treatment comprises administering a focal adhesion kinase (FAK) inhibitor. In certain embodiments, the treatment comprises administering an GCN2 inhibitor and/or Akt inhibitor. In certain embodiments, the treatment comprises administering a RHOT1 inhibitor and/or RHOT2 inhibitor. In certain embodiments, the treatment comprises administering podophyllotoxin, curcumin, NSC319726, Navitoclax, Tariquidar, NVP-LCQ195, SP2509, BMS265246, GCN2-IN-1, BAY-1816032, CGK733, FII-2, and/or Akt inhibitor VIII. In certain embodiments, the treatment comprises administering a chemotherapy, radiation therapy, immunotherapy, hormone therapy, stem cell or bone marrow transplant, and/or surgical resection. In certain embodiments, the treatment comprises FOLFIRINOX or FOLFOXIRI.

In another aspect, a composition comprising a collection of probes, primers, and/or antibodies suitable for detection of the expression and/or expression levels of a collection of genes, or their products, in a tumor sample is provided. The collection of probes, primers, and/or antibodies is suitable for detection of the expression and/or expression levels of a collection of genes, or their products, in a tumor sample, wherein the collection of genes comprises at least eleven of Mic60, XAF1, IGFBP3, MX2, IFIT1, ISG15, OAS3, CXCL10, SERPINE1, CXCL11, CXCL3, MMP13, IFIT3, IFITM1, IFIT1, OAS1, IFI35, MX1, ISG15, IFI6, IFITM2, TAP1, STAT1, IFITM3, STAT2, PSMB8, IRF1, IFNB1, IRF9, IFNGR1, RELA, BAK1, IFNGR2, MED14, IL18, IL1α, IGFBP7, IFIT2, OAS2, MX1, IL6, IL8, CXCL2, CCL26, CCL20, VEGFC, IL15, CXCL6, CXCL1, SERPINB2, ATF5, CD55, KITLG, IGFBP6, CD276, LAMB2, ANG, ETS2, INHBA, AXL, AREG, RELA, and/or MIF. In certain embodiments, the collection of genes consists of XAF1, IGFBP3, MX2, IFIT1, ISG15, OAS3, CXCL10, SERPINE1, CXCL11, CXCL3, and MMP13.

Other aspects and advantages of the invention will be readily apparent from the following detailed description of the invention.

### DESCRIPTION OF THE FIGURES

FIG. 1A-FIG. 1H show the requirement of Mic60 for tumor cell proliferation. (FIG. 1A) PC3 cells transduced with pLKO or shMic60 were labeled with CellTrace violet and analyzed by flow cytometry. The MFI of three independent experiments (mean±SD) are shown. (FIG. 1B) PC3 cells transfected with siCtrl or siMic60 were analyzed by propidium iodide (PI) staining and flow cytometry after 7 d. The percentage of cells in the various cell cycle phases (mean±SD) is shown (n=4). (FIG. 1C) PC3 (Top) or PRAD DU145 (Bottom) cells as in B were analyzed for cell proliferation by direct cell counting at the indicated time intervals. Mean±SD (n=4 to 5). PC3, *P=0.02; ** P=0.008. DU145, *P=0.01 to 0.02; *** P=0.0001. ns, not significant. (FIG. 1D) PC3 cells as in A were analyzed for colony formation (mean±SD) by crystal violet staining and light microscopy. Representative images are shown (n=6). P<0.0001. (FIG. 1E) PC3 cells as in FIG. 1A (Top) or wild-type (WT) or Mic60 knockout (KO) PC3 cells (Bottom) were injected subcutaneously in immunocompromised NSG mice and flank tumor growth was quantified at the indicated time intervals. Each line corresponds to an individual tumor. The mean±SD of tumor growth (mm3) at d 35 is indicated (n=6 to 8). P<0.0001. (FIG. 1F) PC3 cells transfected with siCtrl or siMic60 were labeled for oxidized lipids (Bodipy) and analyzed after 72 h (Top) or 96 h (Bottom) by flow cytometry. MFIs of a representative experiment are indicated (n=3). Gray shades, unstained cells. (FIG. 1G) PC3 cells as in G were analyzed for DEVDase activity/PI staining by multiparametric flow cytometry. The percentage of cells (mean±SD) in early (Bottom right) or late (Top right) apoptosis are indicated (n=3). (FIG. 1H) PC3 cells as in FIG. 1F were analyzed for cell death at the indicated time intervals by Trypan blue exclusion and direct cell counting. Mean±SD (n=2 to 3).

FIG. 2A-FIG. 2D show a Mic60 transcriptome in cancer. (FIG. 2A) PC3 cells transfected with siCtrl or siMic60 were analyzed by RNA-Seq, and relative fold changes in gene expression (false discovery rate [FDR]<5%) were visualized in a heatmap. (FIG. 2B) Enrichment analysis of genes significantly different (FDR<5%) between siCtrl and siMic60 from RNA-Seq results. Ingenuity pathway analysis (Top, canonical pathways) of pathways activated or inhibited after Mic60 knockdown. The number of genes affected and activation Z-scores are indicated. The Fisher exact test was used to test the significance of the enrichment of the SASP-like gene group, and the P value with fold enrichment of SASP-like vs. non-SASP gene products among the list of significant genes is shown. (Bottom) (FIG. 2C) The complete Mic60 transcriptome comprising 52 IFN/SASP-like genes was averaged across all TCGA tumors, and average levels were examined for differential expression in cancer vs. normal samples (ratio). The number of tumor and normal tissue samples is indicated per each condition. The broken line indicates ratio of 1, and the scale indicates significance by P value. (FIG. 2D) PC3 cells transduced with pLKO or shMic60 were transfected with siCtrl or STING-directed siRNA (siSTING) and analyzed for the expression of representative genes in the Mic60 transcriptome by RT-PCR. Representative experiment is shown (n=2).

FIG. 3A-FIG. 3G show Mic60 regulation of tumor cell motility and metastasis. (FIG. 3A) PC3 cells transduced with pLKO or shMic60 were analyzed for circularity (Top; 1=perfect sphere) and surface area (Bottom). Each symbol corresponds to an individual determination. Min to max values are indicated (pLKO, n=36; shMic60, n=39). *** P<0.0001 to 0.0009. (FIG. 3B) PC3 cells as in FIG. 3A were analyzed by Western blotting. (FIG. 3C) PC3 cells transduced with pLKO or shMic60 were analyzed for single-cell motility in 2D contour plots by time-lapse video microscopy. Each tracing corresponds to the movements of an individual cell (pLKO, n=90; shMic60, n=94). The average speed of cell movements (velocity [Vel], μm/min) and distance traveled (Dis; μm) are indicated. Mean±SD (n=3). P<0.0001. The cutoff velocities for slow-moving (red, <0.34 μm/min) or fast-moving (black, >34 μm/min) cells are indicated. (FIG. 3D) PC3 cells as in FIG. 3A or WT or Mic60 KO PC3 cells were analyzed for invasion across Matrigel-coated inserts and quantified. Mean±SD (n=4 to 5). *** P<0.0001 to 0.0002. (FIG. 3E) PC3 cells as in FIG. 3A were transfected with vector or Mic60 cDNA and analyzed for Matrigel invasion. Two independent experiments were conducted (n=21 to 43). (FIG. 3F) PC3 cells as in FIG. 3A were injected into the spleen of NSG mice, and liver samples harvested after 11 d were stained with hematoxylin/eosin, and the number (FIG. 3F, Top) and surface area (FIG. 3F, Bottom) of metastatic foci were quantified by morphometry. Mean±SD (n=30 to 36). *** P<0.0001. Yellow circles, liver metastases. Scale bar, 200 μm. (FIG. 3G) Matched patient samples of LUAD representative of normal parenchyma (N), primary tumor (P), or metastasis (M) were analyzed for differential Mic60 expression by RT-PCR. Top, individual sample analysis; Bottom, mean±SD.

FIG. 4A-FIG. 4I show therapeutic vulnerability of GCN2/Akt signaling exposed by Mic60 depletion. (FIG. 4A and FIG. 4B) PC3 cells transduced with pLKO or shMic60 were analyzed in a high-throughput drug screening (FIG. 4A), and candidate compounds with differential tumor cell killing in shCtrl- or shMic60-transduced cultures (FIG. 4B) were analyzed based on IC50 values (nM). (FIG. 4C) PC3 cells transfected with siMic60 were analyzed for differential ISR gene expression by RNA-Seq. A representative experiment is shown. (FIG. 4D) PC3 cells transfected with siCtrl or siMic60 were analyzed by Western blotting. p, phosphorylated. (FIG. 4E) PC3 cells as in FIG. 4A and FIG. 4B were transfected with ATF4-mScarlet nuclear reporter gene and analyzed by flow cytometry in two independent experiments (Exp). The percentage of cells in gated regions is indicated. (FIG. 4F) Kaplan-Meier survival curves for differential expression of DDIT3 or ATF4 in Mic60-low GBM or pan-kidney cancer cohort comprising kidney chromophobe, kidney renal clear cell carcinoma, and kidney renal papillary cell carcinoma (KIPAN) in the Human Protein Atlas database. The number of patients per each condition and P values are indicated. (FIG. 4G) PC3 cells as in FIG. 4A and FIG. 4B were incubated with vehicle (Veh) or GCN2-IN-1 small-molecule inhibitor to GCN2 and analyzed for Annexin V/PI staining by multiparametric flow cytometry. The percentage of cells in each quadrant is indicated in a representative experiment (n=2). (FIG. 4H) PC3 cells were transfected with siCtrl or GCN2-directed siRNA (siGCN2) and analyzed by Western blotting. (FIG. 4I) PC3 cells were transfected with siCtrl or GCN2-directed siRNA (siGCN2) and analyzed for cell death by Trypan blue exclusion and light microscopy after 72 h. Mean±SD.

FIG. 5A-FIG. 5C show differential expression of a Mic60-low gene signature in cancer. (FIG. 5A) TCGA tumor sets with RNA-seq data from matching normal tissues were examined for differential expression of the 11-gene Mic60-low gene signature in cancer vs. normal samples (ratio) by Wilcoxon rank-sum test. The number of tumor and normal tissue samples is indicated per each condition. Broken line, ratio of 1; red color scale, significance p value; ns, not significant. (FIG. 5B) Differential expression of the 11-gene Mic60-low gene signature in patient-derived tumor-free normal brain margins (MG), LGG or GBM by qPCR. The expression intensity of each gene is visualized in a heatmap. (FIG. 5C) Kaplan-Meier survival curves of high vs. low expression of the Mic60-low gene signature in TCGA datasets of GBM-LGG, kidney renal clear cell carcinoma (KIRC), kidney renal papillary cell carcinoma (KIRP) and uveal melanoma (UVM). The number of patients per condition, p value and hazard ratio (HR) are indicated.

FIG. 6A-FIG. 6D show characterization of Mic60 in pancreatic cancer. (FIG. 6A) PDAC cell lines PANC-1 (top) or CAPAN-2 (bottom) were transfected with control non-targeting siRNA (siCtrl) or Mic60-directed siRNA (siMic60) and analyzed by Western blotting. (FIG. 6B) The conditions are as in (FIG. 6A) and transfected cell lines were analyzed for differential expression of the indicated genes in the Mic60-low gene signature by qPCR. Mean±SEM (n=3). *, p=0.01-0.04; **, p=0.001-0.004; ***, p<0.0001. (FIG. 6C and FIG. 6D) The conditions are as in FIG. 6A and transfected PANC-1 (FIG. 6C) or CAPAN-2 (FIG. 6D) cells were analyzed for cell motility and representative images of DAPI-stained nuclei of migrated or invaded cells were visualized by fluorescence microscopy and quantified. Mean±SEM (n=3). ***, p<0.0001.

FIG. 7A-FIG. 7E show a Mic60-low gene signature and PDAC risk. (FIG. 7A) Kaplan-Meier plots of PDAC overall survival (OS), disease-specific survival (DSS) and disease-free status (DFS) determined by univariate Cox regression tests in the TCGA datasets (N=32). (FIG. 7B) Expression of the 11-gene Mic60-low gene signature in PDAC molecular subtypes by Wilcoxon rank sum test (Moffitt) and Kruskal Wallis test (Collison, Bailey). QM, quasimesenchymal; ADEX, aberrantly differentiated endocrine exocrine; Immunogen, immunogenic; Progen, pancreatic progenitor. (FIG. 7C) Modulation of inflammation-associated markers, IFNγ, PD-L1, PD1 and T cells all by Wilcoxon test. (FIG. 7D) Waterfall plot of tumor size changes in PDAC patients treated with FOLFIRINOX by Wilcoxon test. non-eval, non-evaluable. (FIG. 7E) Kaplan-Meier survival curve of PDAC overall survival (95% CI: 0.533-0.9118). Analyses were carried out by single-sample Gene Set Enrichment Analysis (ssGSEA) with Mic60-low gene signature high (>0.024) or low (<=0.024) and Wilcoxon test.

### DETAILED DESCRIPTION OF THE INVENTION

The role of mitochondria in cancer is not understood. It is shown herein that many human tumors have decreased levels of the inner membrane mitochondrial protein, Mic60 (or IMMT), an essential component of the MICOS complex that controls cristae and membrane assembly. Despite catastrophic loss of mitochondrial integrity, collapse of bioenergetics and oxidative damage, tumors with deletion/depletion of Mic60 escape cell death, slow down cell proliferation, and express a unique gene signature of innate immunity and cytokine/chemokine signaling. Provided herein is an optimized mitochondrial “ghost” gene signature of Mic60 depletion that is broadly overexpressed in tumors compared to normal tissues and associates with aggressive molecular subtypes, treatment failure and shortened patient survival in pancreatic cancer. Mechanistically, Mic60-depleted tumors show heightened mitochondrial fission, increased mitochondrial-fueled cell motility and invasion, and greater metastatic dissemination, in vivo. Further, the small molecule drug screen described herein, uncovers adaptive activation of a GCN2-Akt integrated stress response as a survival mechanism in Mic60-depleted tumors and actionable therapeutic target. Accordingly, acutely damaged, “ghost” mitochondria are common in cancer, drive aggressive disease via adaptive inter-organelle communication and provide actionable therapeutic vulnerabilities.

It is to be noted that the term “a” or “an” refers to one or more. As such, the terms “a” (or “an”), “one or more,” and “at least one” are used interchangeably herein.

While various embodiments in the specification are presented using “comprising” language, under other circumstances, a related embodiment is also intended to be interpreted and described using “consisting of” or “consisting essentially of” language. The words “comprise”, “comprises”, and “comprising” are to be interpreted inclusively rather than exclusively. The words “consist”, “consisting”, and its variants, are to be interpreted exclusively, rather than inclusively.

As used herein, the term “about” means a variability of 10% from the reference given, unless otherwise specified.

“Upregulate” and “upregulation”, as used herein, refer to an elevation in the level of expression of a product of one or more genes in a cell or the cells of a tissue or organ.

“Inhibit” or “downregulate”, as used herein refer to a reduction in the level of expression of a product of one or more genes in a cell or the cells of a tissue or organ.

By the general terms “blocker”, “inhibitor”, or “antagonist” is meant an agent that inhibits, either partially or fully, the activity or production of a target molecule, e.g., as used herein, e.g., Akt. In particular, these terms refer to a composition or compound or agent capable of decreasing levels of gene expression, mRNA levels, protein levels or protein activity of the target molecule. Illustrative forms of antagonists include, for example, proteins, polypeptides, peptides (such as cyclic peptides), antibodies or antibody fragments, peptide mimetics, nucleic acid molecules, antisense molecules, ribozymes, aptamers, RNAi molecules, and small organic molecules. Illustrative non-limiting mechanisms of antagonist inhibition include repression of ligand synthesis and/or stability (e.g., using, antisense, ribozymes or RNAi compositions targeting the ligand gene/nucleic acid), blocking of binding of the ligand to its cognate receptor (e.g., using anti-ligand aptamers, antibodies or a soluble, decoy cognate receptor), repression of receptor synthesis and/or stability (e.g., using, antisense, ribozymes or RNAi compositions targeting the ligand receptor gene/nucleic acid), blocking of the binding of the receptor to its cognate receptor (e.g., using receptor antibodies) and blocking of the activation of the receptor by its cognate ligand (e.g., using receptor tyrosine kinase inhibitors). In addition, the blocker or inhibitor may directly or indirectly inhibit the target molecule.

A “subject” is a mammal, e.g., a human, mouse, rat, guinea pig, dog, cat, horse, cow, pig, or non-human primate, such as a monkey, chimpanzee, baboon, or gorilla. The term “patient” may be used interchangeably with the term subject. In one embodiment, the subject is a human. The subject may be of any age, as determined by the health care provider. In certain embodiments described herein, the patient is a subject who has previously been diagnosed with cancer. The subject may have been treated for cancer previously, or is currently being treated for cancer. In one embodiment, the subject is experiencing stress which has an impact on the beta-adrenergic signaling pathway.

“Sample” as used herein means any biological fluid or tissue that contains blood cells, immune cells and/or cancer cells. In one embodiment, the sample is whole blood. In another embodiment, the sample is plasma. Other useful biological samples include, without limitation, peripheral blood mononuclear cells, plasma, saliva, urine, synovial fluid, bone marrow, cerebrospinal fluid, vaginal mucus, cervical mucus, nasal secretions, sputum, semen, amniotic fluid, bronchoscopy sample, bronchoalveolar lavage fluid, and other cellular exudates from a patient having cancer. Such samples may further be diluted with saline, buffer or a physiologically acceptable diluent. Alternatively, such samples are concentrated by conventional means.

The term “cancer” or “proliferative disease” as used herein means any disease, condition, trait, genotype, or phenotype characterized by unregulated cell growth or replication as is known in the art. A “cancer cell” is cell that divides and reproduces abnormally with uncontrolled growth. This cell can break away from the site of its origin (e.g., a tumor) and travel to other parts of the body and set up another site (e.g., another tumor), in a process referred to as metastasis. A “tumor” is an abnormal mass of tissue that results from excessive cell division that is uncontrolled and progressive and is also referred to as a neoplasm. Tumors can be either benign (not cancerous) or malignant. The methods described herein are useful for the treatment of cancer and tumor cells, i.e., both malignant and benign tumors. In various embodiments of the methods and compositions described herein, the cancer can include, without limitation, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, acute and chronic lymphocytic and myelocytic leukemia, myeloma, Hodgkin's and non-Hodgkin's lymphoma, and multi-drug resistant cancers. In one embodiment, the cancer is lung cancer. In another embodiment, the cancer is ovarian cancer. In another embodiment, the cancer is prostate cancer. In another embodiment, the cancer is lung cancer. In another embodiment, the cancer is colorectal cancer. In another embodiment, the cancer is brain cancer. In certain embodiments, the cancer is pancreatic ductal adenocarcinoma (PDAC). In certain embodiments, the cancer is glioblastoma (GBM).

“Control” or “control level” as used herein refers to the source of the reference value for the biomarker gene levels as well as the particular panel of control subjects identified in the examples below. In some embodiments, the control subject is a healthy subject with no disease. In another embodiment, the control subject is a patient who has been successfully treated for cancer. In yet other embodiments, the control or reference is the same subject from an earlier time point. In one embodiment, the control or reference level is from a single subject. In another embodiment, the control or reference level is from a population of individuals sharing a specific characteristic. In yet another embodiment, the control or reference level is an assigned value which correlates with the level of a specific control individual or population, although not necessarily measured at the time of assaying the test subject's sample. Selection of the particular class of controls depends upon the use to which the diagnostic/monitoring methods and compositions are to be put by the physician. In certain embodiments, the control or control level is obtained from healthy or normal tissue in a subject with disease. In certain embodiments, the control or control level is obtained from healthy or normal tissue in a subject without disease. In certain embodiments, the control or control level is obtained from a tumor-free margin.

The terms “analog”, “modification”, and “derivative” refer to biologically active derivatives of the reference molecule that retain desired activity as described herein. Preferably, the analog, modification or derivative has at least the same desired activity as the native molecule, although not necessarily at the same level. The terms also encompass purposeful mutations that are made to the reference molecule.

By “fragment” is intended a molecule consisting of only a part of the intact full-length polypeptide sequence and structure. The fragment can include a C terminal deletion, an N terminal deletion, and/or an internal deletion of the native polypeptide. A fragment will generally include at least about 5-10 contiguous amino acid residues of the full length molecule, preferably at least about 15-25 contiguous amino acid residues of the full length molecule, and most preferably at least about 20 50 or more contiguous amino acid residues of the full length molecule, or any integer between 5 amino acids and the full length sequence, provided that the fragment in question retains the ability to elicit the desired biological response, although not necessarily at the same level.

By the term “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule.

The antibody may be a naturally occurring antibody or may be a synthetic or modified antibody (e.g., a recombinantly generated antibody; a chimeric antibody; a bispecific antibody; a humanized antibody; a camelid antibody; and the like). The antibody may comprise at least one purification tag. In a particular embodiment, the framework antibody is an antibody fragment. The term “antibody fragment” includes a portion of an antibody that is an antigen binding fragment or single chains thereof. An antibody fragment can be a synthetically or genetically engineered polypeptide. Examples of binding fragments encompassed within the term “antigen-binding portion” of an antibody include (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment, which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding fragment” of an antibody. These antibody fragments are obtained using conventional techniques known to those in the art, and the fragments can be screened for utility in the same manner as whole antibodies. Antibody fragments include, without limitation, immunoglobulin fragments including, without limitation: single domain (Dab; e.g., single variable light or heavy chain domain), Fab, Fab′, F(ab′)2, and F (v); and fusions (e.g., via a linker) of these immunoglobulin fragments including, without limitation: scFv, scFv2, scFv-Fc, minibody, diabody, triabody, and tetrabody. The antibody may also be a protein (e.g., a fusion protein) comprising at least one antibody or antibody fragment.

The term “derived from” is used to identify the original source of a molecule (e.g., bovine or human) but is not meant to limit the method by which the molecule is made which can be, for example, by chemical synthesis or recombinant means.

By “change in expression” is meant an upregulation of one or more selected genes in comparison to the reference or control; a downregulation of one or more selected genes in comparison to the reference or control; or a combination of certain upregulated genes and down regulated genes.

By “therapeutic reagent” or “regimen” is meant any type of treatment employed in the treatment of cancers with or without solid tumors, including, without limitation, chemotherapeutic pharmaceuticals, biological response modifiers, radiation, diet, vitamin therapy, hormone therapies, gene therapy, surgical resection, etc.

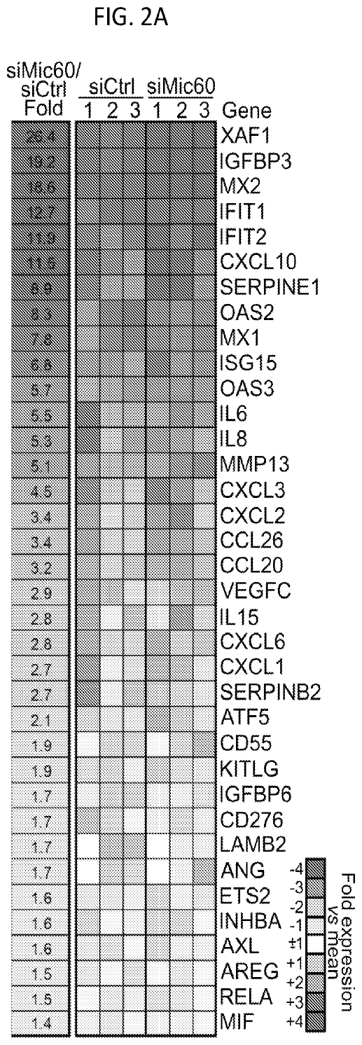

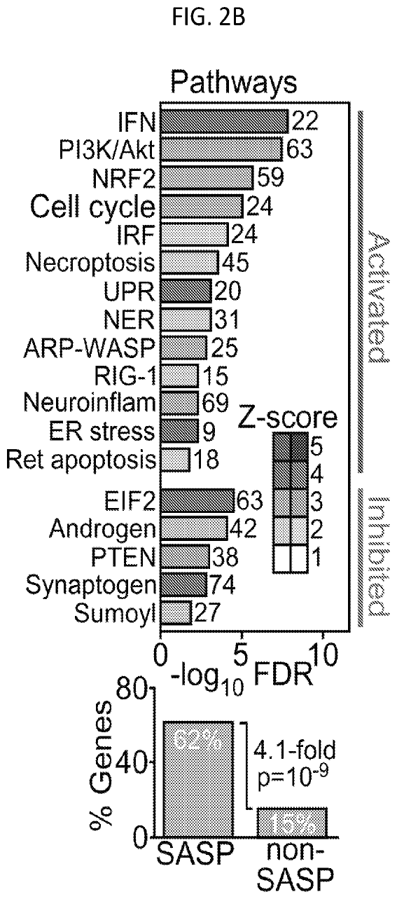

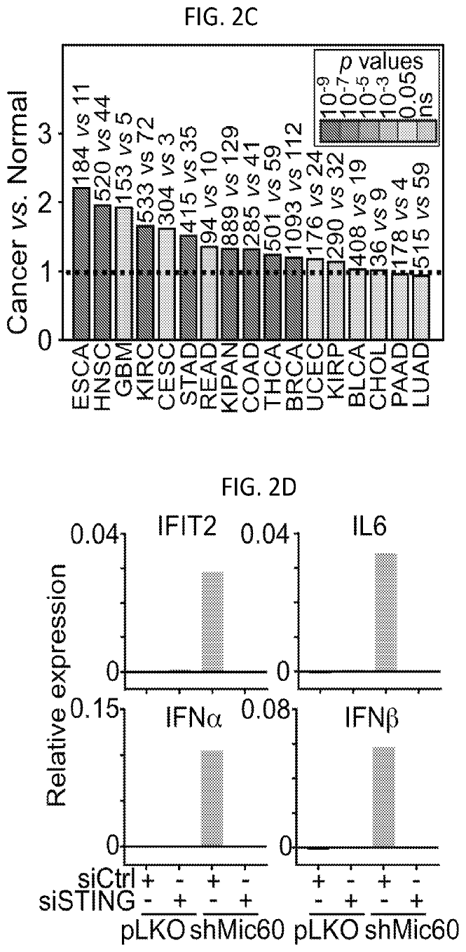

By “target biomarker” or “target biomarker signature” as used herein is meant those proteins/peptides or the genes/transcripts encoding same, the expression of which changes (either in an up-regulated or down-regulated manner) characteristically in the presence of an Mic60-depleted cancer. In one embodiment, at least one target biomarker forms a suitable biomarker signature for use in the methods and compositions. In one embodiment, at least two target biomarkers form a suitable biomarker signature for use in the methods and compositions. In one embodiment, at least three target biomarkers form a suitable biomarker signature for use in the methods and compositions. In one embodiment, at least four target biomarkers form a suitable biomarker signature for use in the methods and compositions. In another embodiment, at least five biomarkers form a suitable biomarker signature for use in the methods and compositions. In one embodiment, at least six target biomarkers form a suitable biomarker signature for use in the methods and compositions. In one embodiment, at least seven target biomarkers form a suitable biomarker signature for use in the methods and compositions. In one embodiment, at least eight target biomarkers form a suitable biomarker signature for use in the methods and compositions. In one embodiment, at least nine target biomarkers form a suitable biomarker signature for use in the methods and compositions. In one embodiment, at least ten target biomarkers form a suitable biomarker signature for use in the methods and compositions. In one embodiment, at least ten target biomarkers form a suitable biomarker signature for use in the methods and compositions. In still further embodiments, at least 9, at least 12, at least 15, at least 20, 30, 40, 50 or at least 60 of the biomarkers including any numbers therebetween identified in Table 1 and/or FIG. 2A form a suitable biomarker signature for the diagnosis of Mic60-depleted cancer. Specific biomarker signatures can include any combination of Mic60-depleted cancer biomarkers employing at least one biomarker from (i) to (xi) identified herein and including all 11 biomarkers in Table 2, as well as other combinations with the biomarkers of Table 1 and/or FIG. 2A. One skilled in the art may readily reproduce the compositions and methods described herein by use of the sequences of the biomarkers, all of which are publicly available from conventional sources, such as GenBank.

TABLE 1
GENE SYMBOL

| | |
|---|---|
| Mic60 | MED14 |
| XAF1 | IL18 |
| IGFBP3 | IL1α |
| MX2 | IGFBP7 |
| IFIT1 | IFIT2 |
| ISG15 | OAS2 |
| OAS3 | MX1 |
| CXCL10 | IL6 |
| SERPINE1 | IL8 |
| CXCL11 | CXCL2 |
| CXCL3 | CCL26 |
| MMP13 | CCL20 |
| IFIT3 | VEGFC |
| IFITM1 | IL15 |
| IFIT1 | CXCL6 |
| OAS1 | CXCL1 |
| IFI35 | SERPINB2 |
| MX1 | ATF5 |
| ISG15 | CD55 |
| IFI6 | KITLG |
| IFITM2 | IGFBP6 |
| TAP1 | CD276 |
| STAT1 | LAMB2 |
| IFITM3 | ANG |
| STAT2 | ETS2 |
| PSMB8 | INHBA |
| IRF1 | AXL |
| IFNB1 | AREG |
| IRF9 | RELA |
| IFNGR1 | MIF |
| RELA | |
| BAK1 | |
| IFNGR2 | |

TABLE 2

| | GENE | SEQ |
|---|---|---|
| GENE NAME | SYMBOL | ID NO: |
| Matrix metallopeptidase 13 | MMP13 | 1 |
| XIAP associated factor 1 | XAF1 | 2 |
| insulin like growth factor binding protein 3 | IGFBP3 | 3 |
| MX dynamin like GTPase 2 | MX2 | 4 |
| interferon induced protein with tetratricopeptide | IFIT1 | 5 |
| repeats 1 | | |
| ISG15 ubiquitin like modifier | ISG15 | 6 |
| 2′-5′-oligoadenylate synthetase 3 | OAS3 | 7 |
| C-X-C motif chemokine ligand 10 | CXCL10 | 8 |
| serpin family E member 1 | SERPINE1 | 9 |
| C-X-C motif chemokine ligand 11 | CXCL11 | 10 |
| C-X-C motif chemokine ligand 3 | CXCL3 | 11 |

The term “microarray” refers to an ordered arrangement of hybridizable array elements, e.g., primers, probes, ligands, on a substrate.

The term “ligand” refers to a molecule that binds to a protein or peptide, and includes antibodies and fragments thereof.

The term “polynucleotide,” when used in singular or plural form, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

As used herein, “labels” or “reporter molecules” are chemical or biochemical moieties useful for labeling a nucleic acid (including a single nucleotide), polynucleotide, oligonucleotide, or protein ligand, e.g., amino acid, peptide sequence, protein, or antibody. “Labels” and “reporter molecules” include fluorescent agents, chemiluminescent agents, chromogenic agents, quenching agents, radionucleotides, enzymes, substrates, cofactors, inhibitors, radioactive isotopes, magnetic particles, and other moieties known in the art. “Labels” or “reporter molecules” are capable of generating a measurable signal and may be covalently or noncovalently joined to an oligonucleotide or nucleotide (e.g., a non-natural nucleotide) or ligand.

As used herein, the term “a therapeutically effective amount” refers an amount sufficient to achieve the intended purpose. For example, an effective amount of an Akt inhibitor is sufficient to inhibit or kill Mic-60 depleted cancer cells. An effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined by a skilled artisan according to established methods in the art.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

Routes of administration include, but are not limited to, intradermal, intramuscular, intraperitoneal, intravenous, subcutaneous, intranasal, epidural, and oral routes. The agent may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

As used herein, “disease”, “disorder”, and “condition” are used interchangeably, to indicate an abnormal state in a subject.

### Target Biomarkers and Biomarker Signatures Useful in the Methods and Compositions

An important regulator of mitochondrial integrity is Mic60, also called Mitofilin or inner mitochondrial membrane protein (IMMT). Mic60 is an essential constituent of a MICOS complex that ensures cristae architecture, organization of respiratory complex(es), outer membrane biogenesis and contact site dynamics. Although a role of this pathway in cancer has not been determined, there is evidence that Mic60 participates in adaptive mitochondrial fitness, including PINK1/Parkin-directed mitophagy, mitochondrial dynamics, especially fission, and modulation of cell death.

As demonstrated herein, many human tumors have decreased levels of Mic60. Provided herein, is an optimized mitochondrial “ghost” gene signature of Mic60 depletion that is broadly overexpressed in tumors compared to normal tissues and associates with aggressive molecular subtypes, treatment failure and shortened patient survival in pancreatic cancer. The “targets” of the compositions and methods of these inventions include, in one aspect, the genes, gene fragments, transcripts and the expression products, including the proteins and fragments thereof, listed in Table 1 and Table 2, which comprise this gene signature. As described in the Examples below, the inventors identified 63 gene transcripts that differed in expression by at least 1.4 fold between Mic60 silenced PC3 cells and control transfected cells. In certain embodiments, superior tests for diagnosing Mic60-depleted cancers utilize at least one of the novel biomarkers. In other embodiments, superior diagnostic tests for evaluating the prognosis of cancer treatment, utilize at least one of the novel biomarkers. In other embodiments, the methods utilize at least two or more of the specific target biomarkers identified herein. In still other embodiments, the methods utilize at least three, four, five, six, seven, eight, nine, ten, eleven or more of the specific target biomarkers identified herein. In certain embodiments, the method employs each of the biomarkers of Table 2. In still other embodiments, at least 12 or more biomarkers of Table 1 will be employed.

### Diagnostic Reagents and Kits

### Labeled or Immobilized Biomarkers or Peptides

In one embodiment, diagnostic reagents for use in the methods of diagnosing, treating and/or evaluating the prognosis of a Mic60-depleted cancer includes one target biomarker identified in Table 1 or Table 2 herein, associated with a detectable label or portion of a detectable label system. In another embodiment, a diagnostic reagent includes one target biomarker identified in Table 1 or Table 2 herein, immobilized on a substrate. In still another embodiment, combinations of such labeled or immobilized biomarkers are suitable reagents and components of a diagnostic kit. Among such immobilized or labeled biomarkers are those selected from the biomarkers:
i. XAF1,
ii. IGFBP3,
iii. MX2,
iv. IFIT1,
V. ISG15,
vi. OAS3,
vii. CXCL10,
viii. SERPINE1,
ix. CXCL11,
X. CXCL3, and
xi. MMP13.

In another aspect, suitable embodiments of such labeled or immobilized reagents include at least 1, 2, 3, 4, 5, 6, 7, 8, 9, 10, or all 11 of biomarkers (i) to (xi).

In another aspect, suitable embodiments of such labeled or immobilized reagents include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or all 63 of the biomarkers in Table 2.

Any combination of labeled or immobilized biomarkers can be assembled in a kit for the purposes of diagnosing, treating and/or evaluating the prognosis of a Mic60-depleted cancer. For example, one embodiment of a kit includes labeled or immobilized reagents (i) through (xi). Another embodiment of a kit includes labeled or immobilized reagents (i) through (viii).

For these reagents, the labels may be selected from among many known diagnostic labels, including those described above. Similarly, the substrates for immobilization may be any of the common substrates, glass, plastic, a microarray, a microfluidics card, a chip or a chamber.

Labeled or Immobilized Ligands that Bind the Biomarkers or Peptides

In another embodiment, the diagnostic reagent is a ligand that binds to a biomarker of any one or more of (i) to (xi) or a fragment thereof, as indicated in Table 1. Such a ligand desirably binds to a protein biomarker or a fragment thereof, and can be an antibody which specifically binds a single biomarker from (i) to (xi). Various forms of antibody, e.g., polyclonal, monoclonal, recombinant, chimeric, as well as fragments and components (e.g., CDRs, single chain variable regions, etc.) may be used in place of antibodies. The ligand itself may be labeled or immobilized.

In another aspect, suitable embodiments of such labeled or immobilized reagents include at least 1, 2, 3, 4, 5, 6, 7, 8, 9, 10, or 11 ligands. Each ligand binds to a single biomarker (i) to (xi).

In another aspect, suitable embodiments of such labeled or immobilized reagents include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 ligands. Each ligand binds to a single biomarker in Table 2.

Any combination of labeled or immobilized biomarker-binding ligands can be assembled in a kit for the purposes of diagnosing, treating and/or evaluating the prognosis of a Mic60-depleted cancer. For example, one embodiment of a kit includes labeled or immobilized reagents that bind to biomarkers (i) through (xi).

Labeled or Immobilized Polynucleotide Oligonucleotides that Hybridize to Genes, Gene Fragments, Gene Transcripts of Other Sequences Encoding the Biomarkers or Peptides

In another embodiment, the diagnostic reagent is a polynucleotide or oligonucleotide sequence that hybridizes to gene, gene fragment, gene transcript or nucleotide sequence encoding a biomarker of any of one or more of (i) to (xi). Such a polynucleotide/oligonucleotide can be a probe or primer, and may itself be labeled or immobilized. In another aspect, suitable embodiments of such labeled or immobilized reagents include at least 1, 2, 3, 4, 5, 6, 7, 8, 9, 10, or 11 polynucleotides/oligonucleotides. Each polynucleotide/oligonucleotide hybridizes to a gene, gene fragment, gene transcript or expression product encoding a single biomarker (i) to (xi).

In another aspect, suitable embodiments of such labeled or immobilized reagents include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 polynucleotides/oligonucleotides. Each polynucleotide/oligonucleotide hybridizes to a gene, gene fragment, gene transcript or expression product encoding a single biomarker in Table 2.

Any combination of labeled or immobilized biomarker-hybridizable sequences can be assembled in a kit for the purposes of diagnosing, treating and/or evaluating the prognosis of a Mic60-depleted cancer. For example, one embodiment of a kit includes labeled or immobilized reagents that hybridize to biomarkers (i) through (xi).

Still other components of the many biomarker signatures that may be formed by various combinations of polynucleotide/oligonucleotide sequences that hybridize to the biomarkers (i) through (xi) associated with detectable labels or immobilized on substrates provide additional diagnostic kits. Still other components include similar reagents that hybridize to biomarkers or fragments thereof as listed in Table 1. In one embodiment, these polynucleotide or oligonucleotide reagent(s) are part of a primer-probe set, and the kit comprises both primers and probe. Each said primer-probe set amplifies a different gene, gene fragment or gene expression product that encodes a different biomarker of any combination of (i) through (xi).

The design of the primer and probe sequences is within the skill of the art once the particular gene target is selected. The particular methods selected for the primer and probe design and the particular primer and probe sequences are not limiting features of these compositions. A ready explanation of primer and probe design techniques available to those of skill in the art is summarized in U.S. Pat. No. 7,081,340, with reference to publicly available tools such as DNA BLAST software, the Repeat Masker program (Baylor College of Medicine), Primer Express (Applied Biosystems); MGB assay-by-design (Applied Biosystems); Primer3 (Steve Rozen and Helen J. Skaletsky (2000) Primer3 on the web for general users and for biologist programmers and other publications).

Thus, a composition for diagnosing, treating and/or evaluating the prognosis of a Mic60-depleted cancer in a mammalian subject as described herein can be a kit containing multiple reagents or one or more individual reagents. For example, one embodiment of a composition includes a substrate upon which the biomarkers, polynucleotides or oligonucleotides, or ligands are immobilized. In another embodiment, the composition is a kit also contains optional detectable labels, immobilization substrates, optional substrates for enzymatic labels, as well as other laboratory items.

The compositions based on the biomarkers selected from Table 2 or Table 1 described herein, optionally associated with detectable labels, can be presented in the format of a microfluidics card, a chip or chamber, or a kit adapted for use with the assays described in the Examples, ELISAs or PCR, RT-PCR or Q PCR techniques described herein.

The selection of the ligands, poly/oligonucleotide sequences, their length, suitable labels and substrates used in the composition are routine determinations made by one of skill in the art in view of the teachings of which biomarkers form signature suitable for the diagnosis, treatment and/or evaluation of the prognosis of a Mic60-depleted cancer.

### Methods for Diagnosing, Treating, and or Evaluating the Prognosis of a Mic60-Depleted Cancer

### Protein Assays

In one embodiment, a method for diagnosing, treating and/or evaluating the prognosis of a Mic60-depleted cancer in a mammalian subject includes measuring in a biological fluid sample of the subject the expression level of a protein or fragment thereof selected from at least one biomarker of Table 1 or Table 2. Alternatively, the method includes measuring a combination of two or more biomarkers of Table 1 or Table 2. In certain embodiments, the method includes measuring a combination of all 11 biomarkers of Table 2. In certain embodiments, the method includes measuring a combination of 2, 3, 4, 5, 6, 7, 8, 9, or 10 biomarkers of Table 2. The method further involves comparing the subject's expression level of the selected biomarker or biomarker fragment with the level of the same protein or peptide in the biological fluid of a reference or control mammalian subject. Changes in expression of the subject's selected biomarker protein or peptide fragment from those of the reference or control correlates with a diagnosis of Mic60-depleted cancer. In certain embodiments, changes in expression of the subject's selected biomarker protein or peptide fragment from those of the reference or control correlates with shortened patient survival. In certain embodiments, changes in expression of the subject's selected biomarker protein or peptide fragment from those of the reference or control correlates with cancer treatment failure.

In this method, a change in expression level of one or more of the selected biomarker proteins in comparison to the control reference may be an increase or decrease in the expression levels of the individual biomarkers. This method may employ any of the suitable diagnostic reagents or kits or compositions described above.

The measurement of the biomarkers in the biological sample may employ any suitable ligand, e.g., antibody (or antibody to any second biomarker) to detect the biomarker protein. Such antibodies may be presently extant in the art or presently used commercially, such as those available as part of commercial antibody ELISA assay kits or that may be developed by techniques now common in the field of immunology.

Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting selected biomarker-antibody complex in applicable assays. Still other labels include fluorescent compounds, radioactive compounds or elements. Preferably, an anti-biomarker antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

Detectable labels for attachment to antibodies useful in diagnostic assays of this invention may be easily selected from among numerous compositions known and readily available to one skilled in the art of diagnostic assays. The biomarker-antibodies or fragments useful in this invention are not limited by the particular detectable label or label system employed. Thus, selection and/or generation of suitable biomarker-antibodies with optional labels for use in this invention is within the skill of the art, provided with this specification, the documents incorporated herein, and the conventional teachings of immunology.

Similarly, the particular assay format used to measure the selected biomarker in a biological sample may be selected from among a wide range of immunoassays, such as enzyme-linked immunoassays, such as those described in the examples below, sandwich immunoassays, homogeneous assays, immunohistochemistry formats, or other conventional assay formats. One of skill in the art may readily select from any number of conventional immunoassay formats to perform this invention.

Other reagents for the detection of protein in biological samples, such as peptide mimetics, synthetic chemical compounds capable of detecting the selected EP biomarker may be used in other assay formats for the quantitative detection of biomarker protein in biological samples, such as high-pressure liquid chromatography (HPLC), immunohistochemistry, etc.

### Nucleic Acid Assays

Still other methods useful in performing the diagnostic steps described herein are known in the art. Such methods include methods based on hybridization analysis of polynucleotides, methods based on sequencing of polynucleotides, proteomics-based methods or immunochemistry techniques. The most commonly used methods known in the art for the quantification of mRNA expression in a sample include northern blotting and in situ hybridization; RNAse protection assays; and PCR-based methods, such as real-time polymerase chain reaction (RT-PCR) or qPCR. Alternatively, antibodies may be employed that can recognize specific DNA-protein duplexes. The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, CA), differential display, amplified fragment length polymorphism (iAFLP), and BeadArray™ technology (Illumina, San Diego, CA) using the commercially available Luminex 100 LabMAP system and multiple color-coded microspheres (Luminex Corp., Austin, Tex.) and high coverage expression profiling (HiCEP) analysis.

Thus, in yet another embodiment, a method for diagnosing, treating and/or evaluating the prognosis of a Mic60-depleted cancer in a mammalian subject involves measuring in a biological fluid sample of the subject the expression level of a gene, gene fragment, gene transcript or expression product of one or more of the biomarkers of Table 1 or Table 2. In some embodiments, the method includes measuring the expression level of the genes, gene fragments, gene transcripts or expression products of a combination of two or more biomarkers of Table 1 or Table 2. In certain embodiments, the method includes measuring the expression level the genes, gene fragments, gene transcripts or expression products of a combination of all 11 biomarkers of Table 2. In certain embodiments, the method includes measuring the expression level the genes, gene fragments, gene transcripts or expression products of a combination of 2, 3, 4, 5, 6, 7, 8, 9 or 10 biomarkers of Table 2. The method further includes comparing the subject's selected biomarker gene, gene fragment, gene transcript or expression product expression level with the level of the same gene, gene fragment, gene transcript or expression product in the biological fluid of a reference or control. Changes in expression of the subject's selected biomarker gene, gene fragment, gene transcript or expression products from those of the reference or control correlates with a diagnosis of Mic60-depleted cancer. In certain embodiments, changes in expression of the subject's selected biomarker gene, gene fragment, gene transcript or expression products from those of the reference or control correlates with shortened patient survival. In certain embodiments, changes in expression of the subject's selected biomarker gene, gene fragment, gene transcript or expression products from those of the reference or control correlates with cancer treatment failure.

In this method, a change in expression level of one or more of the selected biomarker genes, gene fragments, gene transcripts or expression products in comparison to the control reference may be an upregulation or down regulation in the expression of the individual biomarker genes, gene fragments, transcripts or expression products detected. In certain embodiments, a change in expression level of one or more of the selected biomarker genes, gene fragments, gene transcripts or expression products in comparison to the control reference is an upregulation in the expression of the individual biomarker genes, gene fragments, transcripts or expression products detected. These methods may employ any of the suitable diagnostic reagents or kits or compositions described above.

In certain embodiments, an increase in expression level of one or more of the selected biomarker genes, gene fragments, gene transcripts or expression products in comparison to a control reference is detected. In certain embodiments, an increase in expression level of all of the selected biomarker genes, gene fragments, gene transcripts or expression products in comparison to a control reference is detected. In certain embodiments, an increase in expression level of one or more of the selected biomarker genes, gene fragments, gene transcripts or expression products in comparison to a control reference is greater than or equal to 1.5 fold, 1.75 fold, 2.0 fold, 4.0 fold, 5.0 fold, 6.0 fold, 8.0 fold, or 10 fold.

In yet another embodiment, the methods and compositions described herein may be used in conjunction with clinical risk factors to help physicians make more accurate decisions about how to manage patients with Mic60-depleted cancer. Another advantage of these methods and compositions is that diagnosis may occur early.

In another embodiment of the methods described herein, the method includes treatment of the Mic60-depleted cancer. In one embodiment, the method includes administering an effective amount of Gamitrinib. As used herein, the term “Gamitrinib” refers to any one of a class of geldanamycin (GA)-derived mitochondrial matrix inhibitors. Gamintrinibs contain a benzoquinone ansamycin backbone derived from the Hsp90 inhibitor 17-(allylamino)-17-demethoxygeldanamycin (17-AAG), a linker region on the C17 position, and a mitochondrial targeting moiety, either provided by 1 to 4 tandem repeats of cyclic guanidinium (for example, a tetraguanidinium (G4), triguanidinium (G3), diguanidinium (G2), monoguanidinium (G1), or triphenylphosphonium moiety (Gamitrinib-TPP-OH). For example, Gamitrinib-G4 refers to a Gamitrinib in which a tetraguanidinium moiety is present. For example, Gamitrinib-TPP refers to a Gamitrinib in which a triphenylphosphonium moiety is present. Also throughout this application, the use of the plural form “Gamitrinibs” indicates one or more of the following: Gamitrinib-G4, Gamitrinib-G3, Gamitrinib-G2, Gamitrinib-G1, and Gamitrinib-TPP or Gamitrinib-TPP-OH. Gamitrinib is a small molecule inhibitor of Hsp90 and TRAP-1 ATPase activity, engineered to selectively accumulate in mitochondria. In one embodiment, the Gamintrinib is Gamitrinib-TPP-OH. In another embodiment, the Gamitrinib is Gamitrinib-G4. See, e.g., United States Patent Publication No. 2009/0099080, which is hereby incorporated by reference in its entirety.

In one embodiment, the method includes administering an effective amount of an inhibitor of Akt. In another embodiment, the method includes administering an effective amount of an inhibitor of GCN2. In another embodiment, the method includes administering an effective amount of an inhibitor of FAK. In yet another embodiment, the method includes administering an effective amount of an inhibitor of RHOT1. In yet another embodiment, the method includes administering an effective amount of an inhibitor of RHOT2. Inhibitors include small molecule inhibitors, shRNA, and siRNA. In one embodiment, the FAK inhibitor is FAKi (VS-6062) GSK2256098, BI 853520, SK2256098, VS-4718, or VS-6063. In another embodiment, the GCN2 inhibitor is GCN2-IN-1. In another embodiment, the Akt inhibitor is Akt inhibitor VIII, MK2206. In some embodiments, the treatment comprises administering podophyllotoxin, curcumin, NSC319726, Navitoclax, Tariquidar, NVP-LCQ195, SP2509, BMS265246, GCN2-IN-1, BAY-1816032, CGK733, FII-2, and/or Akt inhibitor VIII. In some embodiments, the treatment comprises administering FOLFIRINOX. In some embodiments, the treatment comprises administering FOLFOXIRI.

In one embodiment, the effective amount of the inhibitor or treatment is an amount ranging from about 0.01 mg/ml to about 10 mg/ml, including all amounts therebetween and end points. In one embodiment, the effective amount of the inhibitor or treatment is about 0.1 mg/ml to about 5 mg/ml, including all amounts therebetween and end points. In another embodiment, the effective amount of the inhibitor or treatment is about 0.3 mg/ml to about 1.0 mg/ml, including all amounts therebetween and end points. In another embodiment, the effective amount of the inhibitor or treatment is about 0.3 mg/ml. In another embodiment, the effective amount of the inhibitor or treatment is about 0.4 mg/ml. In another embodiment, the effective amount of the inhibitor or treatment is about 0.5 mg/ml. In another embodiment, the effective amount of the inhibitor or treatment is about 0.6 mg/ml. In another embodiment, the effective amount of the inhibitor or treatment is about 0.7 mg/ml. In another embodiment, the effective amount of the inhibitor or treatment is about 0.8 mg/ml. In another embodiment, the effective amount of the inhibitor or treatment is about 0.9 mg/ml. In another embodiment, the effective amount of the inhibitor or treatment is about 1.0 mg/ml.

In one embodiment, the effective amount of the inhibitor or treatment is an amount ranging from about 1 μM to about 2 mM, including all amounts therebetween and end points. In one embodiment, the effective amount of the inhibitor or treatment is about 10 μM to about 100 μM, including all amounts therebetween and end points. In another embodiment, the effective amount of the inhibitor or treatment is about 5 μM. In another embodiment, the effective amount of the inhibitor or treatment is about 10 M. In another embodiment, the effective amount of the inhibitor or treatment is about 20 μM. In another embodiment, the effective amount of the inhibitor or treatment is about 50 μM. In another embodiment, the effective amount of the inhibitor or treatment is about 100 μM. In another embodiment, the effective amount of the inhibitor or treatment is about 200 μM. In another embodiment, the effective amount of the inhibitor or treatment is about 300 μM. In another embodiment, the effective amount of the inhibitor or treatment is about 400 μM. In another embodiment, the effective amount of the inhibitor or treatment is about 500 μM. In another embodiment, the effective amount of the inhibitor or treatment is about 600 μM. In another embodiment, the effective amount of the inhibitor or treatment is about 700 μM. In another embodiment, the effective amount of the inhibitor or treatment is about 800 μM. In another embodiment, the effective amount of the inhibitor or treatment is about 900 μM. In another embodiment, the effective amount of the inhibitor or treatment is about 1 mM. In another embodiment, the effective amount of the inhibitor or treatment is about 1.25 mM. In another embodiment, the effective amount of the inhibitor or treatment is about 1.5 mM. In another embodiment, the effective amount of the inhibitor or treatment is about 1.75 mM. In another embodiment, the effective amount of the inhibitor or treatment is about 2 mM.

Other chemotherapeutic agents (e.g., anti-cancer agents) are well known in the art and include, but are not limited to, anthracenediones (anthraquinones) such as anthracyclines (e.g., daunorubicin (daunomycin; rubidomycin), doxorubicin, epirubicin, idarubicin, and valrubicin), mitoxantrone, and pixantrone; platinum-based agents (e.g., cisplatin, carboplatin, oxaliplatin, satraplatin, picoplatin, nedaplatin, triplatin, and lipoplatin); tamoxifen and metabolites thereof such as 4-hydroxytamoxifen (afimoxifene) and N-desmethyl-4-hydroxytamoxifen (endoxifen); taxanes such as paclitaxel (taxol) and docetaxel; alkylating agents (e.g., nitrogen mustards such as mechlorethamine (HN2), cyclophosphamide, ifosfamide, melphalan (L-sarcolysin), and chlorambucil); ethylenimines and methylmelamines (e.g., hexamethylmelamine, thiotepa, alkyl sulphonates such as busulfan, nitrosoureas such as carmustine (BCNU), lomustine (CCNLJ), semustine (methyl-CCN-U), and streptozoein (streptozotocin), and triazenes such as decarbazine (DTIC; dimethyltriazenoimidazolecarboxamide)); antimetabolites (e.g., folic acid analogues such as methotrexate (amethopterin), pyrimidine analogues such as fluorouracil (5-fluorouracil; 5-FU), floxuridine (fluorodeoxyuridine; FUdR), and cytarabine (cytosine arabinoside), and purine analogues and related inhibitors such as mercaptopurine (6-mercaptopurine; 6-MP), thioguanine (6-thioguanine; 6-TG), and pentostatin (2′-deoxycofonnycin)); natural products (e.g., vinca alkaloids such as vinblastine (VLB) and vincristine, epipodophyllotoxins such as etoposide and teniposide, and antibiotics such as dactinomycin (actinomycin D), bleomycin, plicamycin (mithramycin), and mitomycin (mitomycin Q); enzymes such as L-asparaginase; biological response modifiers such as interferon alpha); substituted ureas such as hydroxyurea; methyl hydrazine derivatives such as procarbazine (N-methylhydrazine; MIH); adrenocortical suppressants such as mitotane (o,p′-DDD) and aminoglutethimide; analogs thereof derivatives thereof and combinations thereof.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not intended to limit the present invention.

### Example 1: Materials and Methods

### Patient Samples

Primary patient samples arranged in a universal TMA were examined for differential Mic60 expression by IHC. Archival tissues and clinical records were obtained from Fondazione Istituto di Ricovero e Cura a Carattere Scientifico (IRCCS) Ca' Granda Hospital in Milan, Italy, under a protocol approved by the Institutional Review Boards (IRBs) of Fondazione IRCCS Ca' Granda-Ospedale Maggiore Policlinico (code 179/2013). Because of the retrospective nature of this study and the use of data anonymization practices, the need for written informed consent was waived. For the glioma series, fresh-frozen material was available from Fondazione IRCCS Ca' Granda Hospital. Clinically annotated patient samples with a confirmed histologic diagnosis of PDAC (n=5) were obtained from the archival database of the Department of Pathology at Yale New Haven Hospital upon approval from the Yale University IRB and examined for intratumoral heterogeneity of Mic60 expression by IHC.

Primary patient samples with histologically confirmed diagnosis of normal brain parenchyma (tumor-free surgical margins, N=5), low grade gliomas (LGG, N=4, oligodendroglioma, astrocytoma) and glioblastoma (GBM, N=6) were examined for differential expression of the Mic60-low gene signature by qPCR. For the glioma series, fresh-frozen material was available from Fondazione IRCCS Ca' Granda Hospital. Clinically-annotated patient samples with confirmed histologic diagnosis of PDAC (N=5) were obtained from the archival database of the Department of Pathology at Yale New Haven Hospital upon approval from the Yale University Institutional Review Board and examined for Mic60 expression by immunohistochemistry (IHC). All methods were performed in accordance with the relevant guidelines and regulations using fully de-identified patient material.

### Proteomics

Immune complexes of Mic60 or nonbinding immunoglobulin G (IgG) were precipitated from PC3 cells and separated by sodium dodecyl sulphate gel electrophoresis for ˜5 mm followed by fixing and staining with colloidal Coomassie. The region of the gel-containing proteins was excised and digested with trypsin. Tryptic peptides were analyzed by liquid chromatography-tandem mass spectrometry (LC-MS/MS) on a Q Exactive high-field (HF) mass spectrometer (Thermo Scientific) coupled with a Nano-ACQUITY ultra performance liquid chromatography (UPLC) system (Waters). Samples were injected onto a UPLC Symmetry trap column (180-μm inner diameter [i.d.]×2 cm packed with 5-μm C18 resin; Waters), and tryptic peptides were separated by RP-HPLC on a BEH C18 nanocapillary analytical column (75-μm i.d.×25 cm, 1.7-μm particle size; Waters) using a 90-min gradient. Eluted peptides were analyzed in data-dependent mode where the mass spectrometer obtained full MS scans from 400 to 2,000 m/z at 60,000 resolution. Full scans were followed by MS/MS scans at 15,000 resolution on the 20 most abundant ions. Peptide match was set as preferred, and the exclude isotopes option and charge-state screening were enabled to reject singly and unassigned charged ions. MS/MS spectra were searched using MaxQuant 1.6.5.0 (57) against the UniProt human protein database (October 2017). MS/MS spectra were searched using full tryptic specificity with up to two missed cleavages, static carbamidomethylation of Cys, variable oxidation of Met, and variable protein N-terminal acetylation. Consensus identification lists were generated with false discovery rates of 1% at protein and peptide levels. Undetected protein intensity values of 0 were floored to the value of 106 (minimum nonzero detected intensity was 1,233,300), and a total of 1,534 detected proteins were taken for further annotation analysis. Proteins were then annotated as mitochondrial related using the MitoCarta 2.0 database, and 119 mitochondrial proteins detected with at least 5 peptides at an intensity over 10-fold versus IgG control were considered as Mic60-associated proteins.

### Mitochondrial Time-Lapse Videomicroscopy

Cells (2× 10+) growing on high optical quality glass-bottom 35-mm plates (MatTek Corporation) were incubated with 100 nM Mitotracker Deep Red FM dye for 1 h and imaged on a Leica TCS SP8× inverted laser scanning confocal microscope using a 63× 1.40 NA oil objective.

### Single-Cell Motility

Cells (2× 10+) were seeded in 4-well Ph+ Chambers (Ibidi) in complete growth medium and allowed to attach for 16 h at 37° C. Time-lapse videomicroscopy was performed over 10 h, with a time-lapse interval of 10 min. Stacks were imported into Image J Fiji software for analysis, and at least 10 to 20 cells per condition were tracked using the Manual Tracking plugin for Image J Fiji. Tracking data were exported into the Chemotaxis and Migration Tool v. 2.0 (Ibidi) for graphing and calculation of the mean and SD of speed and accumulated distance of movement. For directional cell migration, wounds were made in monolayers of PC3 cells using a 10-μL pipette tip. Cell debris were washed off, and cultures were maintained in complete growth medium containing 10% fetal bovine serum at 37° C. and 5% CO2. Time-lapse imaging of migrating cells was performed using a TE300 inverted microscope (Nikon) equipped with an incubator set at 37° C., 5% CO2, and 95% relative humidity. Each image was acquired using a 10× objective of the same fields at each 10-min interval for a total of 24 h.

### Small-Molecule Drug Screening

Cell viability screening against the MedChem Express anti-cancer library (1,820 compounds) was performed using CellTiterGlo (Promega). PC3 cells stably transduced with pLKO or Mic60-directed shRNA (shMic60) were maintained in complete media, trypsinized, and plated (500 cells/well) in 40 μL of complete medium the day before the experiment in white, clear-bottom 384-well plates. A total of 50 nL of test compound was added to each well using the Janus MDT Nanohead (Perkin-Elmer). Each compound was screened at a final concentration of 10, 1, 0.1, and 0.01 μM. After a 72-h incubation at 37° C. in the presence of 5% CO2, 20 μL of the CellTiterGlo reagent was added to each well. After 15 min, luminescence was measured using the Envision Multimode plate reader (PerkinElmer). The raw data were normalized to % inhibition, where 0% is the relative light units (RLU) in the presence of dimethylsulfoxide, and 100% is the RLU in the presence of 1 μM bortezomib. Estimated half maximal inhibitory concentration (IC50) values for each compound were determined using nonlinear regression fits on the data to a one-site binding model in XlFit (ID Business Solutions Ltd. [IDBS]). Because only 4 data points were used in this calculation, the top and bottom of the curve was fixed to 100% and 0%, respectively, with a constant slope value of 1.

### Cell Culture Experiments

Human PDAC cell lines PANC-1 and CAPAN-2 (American Type Culture Collection, Manassas, VA) were transfected with control non-targeting siRNA or Mic60-directed siRNA in the presence of 25 nM Lipofectamine RNAiMAX (Invitrogen) at a 1:1 ratio (vol siRNA 20 μM/vol Lipofectamine RNAiMAX), as described. Transfected cells were validated for Mic60 knockdown by Western blotting of total cell extracts prepared in 150 mM NaCl, 1% Triton X-100, 0.5% sodium deoxycholate, 0.1% SDS, 50 mM Tris, pH 8.0 in the presence of EDTA-free Protease Inhibitor Cocktail (Roche) and Phosphatase Inhibitor Cocktail (Roche). In some experiments, transfected PDAC cells were analyzed for cell migration on 8 μm PET inserts or cell invasion across Matrigel-coated inserts.

mRNA Expression

PANC-1 and CAPAN-2 cells transfected as indicated above were harvested and RNA was immediately extracted using Quick-RNA Microprep (Zymo Research) according to the manufacturer's instructions. cDNA was prepared with High32 Capacity cDNA Reverse Transcription Kit with RNase Inhibitor (ThermoFisher Scientific) and the reverse-transcription reaction performed on a BioRad T100 Thermal Cycler. Quantitative PCR was performed with SYBR™ Select Master Mix (ThermoFisher) on ABI Quant Studio 5 machine (ThermoFisher).

### IHC

Four μm-thick sections from tissue blocks of human PDAC tissue samples were stained with a primary antibody to Mic60 (BD Biosciences) using Benchmark Ultra Roche Ventana Immunostainer (Roche Group, Tucson, AZ) and diaminobenzidine (DAB) as a chromogen. All slides were counterstained with hematoxylin.

### TOGA Analysis

As discovery dataset, log 2-transformed mRNA expression values were downloaded from 33 tumor samples in The Cancer Genome Atlas (TCGA) database of the UCSC Xena browser (Pan-Cancer Atlas Hub). Average expression values of the Mic60-low gene signature were examined in tumor sets with RNA-seq data with matching normal tissues. Multivariate Cox regression was used to determine the effect of age, gender, and stage as co-factors on outcome. Cox regression p values and hazard ratios (HR) between patients based on median Mic60 signature values were obtained.

### COMPASS Cohort

The Comprehensive Molecular Characterization of Advanced Pancreatic Ductal Adenocarcinomas (PDAC) for Better Treatment Selection (COMPASS) trial is a prospective multi-institutional Canadian cohort study. Patient eligibility criteria for the study require a radiologic or histologic diagnosis of locally advanced or metastatic PDAC suitable for combination chemotherapy, and consent to a fresh tumor biopsy prior to treatment start. In terms of eligibility criteria, biopsies can be taken from the primary lesion or any metastatic sites and patients must not have had prior treatment for advanced disease. Treatment decisions are at the discretion of their medical oncologist. Response to therapy is assessed using CT and measured using RECIST 1.1. Demographics and treatment details, including subsequent treatments, are prospectively collected using an electronic MEDIDATA database. The COMPASS trial has been approved by the Institutional Review Board at participating sites (University Health Network, Toronto, Ontario, Canada; MUHC Centre for Applied Ethics, Montreal, Quebec, Canada; and Queen's University Health Sciences and Affiliated Teaching Hospitals Research Ethics Board, Kingston, Ontario, Canada); each patient provided written informed consent prior to study entry. Bioinformatics analysis of the 11-gene Mic60-low gene signature was carried out on transcriptomic data collected from all patients enrolled in the trial from December 2015 until May 2019 and follow-up censored on Aug. 30, 2019 (N=195).

### CPTAC Cohort

The Clinical Proteomic Tumor Analysis Consortium (CPTAC) comprises a total of 140 participants (74 males, 66 females between the age group of 31-85) collected by 11 different tissue source sites from 7 different countries. Clinical data were obtained from tissue source sites and aggregated by an internal Comprehensive Data Resource database that synchronizes with the CPTAC Data Coordinating Center. Demographics, histopathologic information, treatment and patient outcome information were collected and reviewed before deposition into the Proteomic Data Commons (PDC) and Genomic Data Commons (GDC). The cohort consists of 53% male (n=74) and 47% female (n=66). Age distributions [31-40 (2.9%), 41-50 (9.3%), 51-60 (16.4%), 61-70 (42.9%), 71-80 (25.7%), and 81-90 (2.9%)] and stage distributions [I (16.4%), II (42.9%), III (30.0%), and IV (6.4%)] of the patients reflect the general incidence of surgically resected PDAC.

### Example 2: Ghost Mitochondria Drive Metastasis Through Adaptive GCN2/Akt Therapeutic Vulnerability

### Mic60 Expression in Cancer

To study the role of mitochondrial fitness in cancer, we focused on Mic60 as an essential scaffold of organelle integrity and function. Inspection of the Human Protein Atlas database showed that Mic60 expression was highly heterogeneous in cancer, as several tumor types had reduced, increased, or unchanged levels of Mic60 compared to normal tissues. Immunohistochemical (IHC) staining of a universal tumor microarray (TMA; n=5 to 8 cases per tumor type) gave similar results, where Mic60 expression was reduced in colorectal adenocarcinoma (COREAD) and glioblastoma (GBM), unchanged in breast (BRCA) and prostate adenocarcinoma (PRAD), or increased in lung adenocarcinoma (LUAD) compared to adjacent normal tissue. Consistent with these results, Mic60 mRNA levels in The Cancer Genome Atlas (TCGA) database were reduced in GBM and COREAD but prominently upregulated in LUAD. Other potential Mic60-low tumors in this analysis included malignancies of kidney, thyroid, head and neck, and soft tissue, whereas uterine and cervix cancer had higher Mic60 mRNA levels compared to normal tissues. Although breast cancer showed increased Mic60 protein and mRNA in public databases, our TMA analysis did not reach statistical significance. Heterogeneous Mic60 expression was also observed intratumorally. When analyzed in patient samples of pancreatic ductal adenocarcinoma (PDAC), Mic60 expression ranged from focal perinuclear distribution in normal pancreatic acinar cells to disordered, submembranous or linear staining in in situ and invasive neoplastic epithelium to absence in poorly differentiated (basaloid) carcinomas by IHC. Mechanistically, differentiation of patient-derived GBM neurospheres, a process associated with the modulation of stemness and proliferative potential, lowered Mic60 as well as HIF1α mRNA levels.

### Mic60-Dependent Mitochondrial Integrity in Cancer

Next, we examined the function of Mic60 in cancer. Using a proteomics screen in PRAD PC3 cells, we identified 119 high-confidence mitochondrial proteins that associate with Mic60. Bioinformatics analysis of this dataset identified multiple regulators of mitochondrial membrane transport and organization, protein sorting, Ca²⁺ homeostasis, and oxidative phosphorylation. Therefore, we sought to reproduce the phenotype of Mic60-low tumors by generating clones of PC3 or GBM LN229 cells with silencing of Mic60 by short hairpin RNA (shRNA) or CRISPR-Cas9. Small interfering RNA (siRNA) sequences targeting Mic60 were also characterized in PC3 cells, normal diploid fibroblasts, MRC5, breast adenocarcinoma MDA231, and osteosarcoma HT1080 cells.

Using these approaches, silencing of Mic60 caused a catastrophic collapse of mitochondrial integrity in tumor cells, with disassembly of tubular network and cristae organization. This was accompanied by acute mitochondrial damage, characterized by increased outer membrane permeability and depolarization of the inner membrane. As a result, Mic60-depleted tumor cells exhibited decreased oxygen consumption rates, with lower basal and maximal respiration, reduced adenosine triphosphate (ATP) production, and increased phosphorylation of AMPK, a marker of cellular starvation. Despite a modest increase in antioxidant glutathione (GSH), these cells showed acute oxidative stress with a decreased GSH:glutathione disulfide (GSSG) ratio, heightened production of total and mitochondrial reactive oxygen species (ROS), and increased expression of γH2AX, as well as formation of subnuclear γH2AX foci. Consistent with loss of mitochondrial integrity, Mic60-low tumor cells activated quality-control mechanisms of autophagy with punctate GFP-LC3 staining, processing of LC3 to a lipidated form, and upregulation of p62, i.e., sequestosome. Mitophagy was also induced in these settings, as judged by increased MitoKeima red fluorescence reporter activity, loss of mitochondrial mass, and degradation of mitochondrial outer membrane proteins. Silencing of p62 was insufficient to restore outer membrane proteins or mitochondrial mass after Mic60 depletion.

### Requirement of Mic60 for Tumor Cell Proliferation

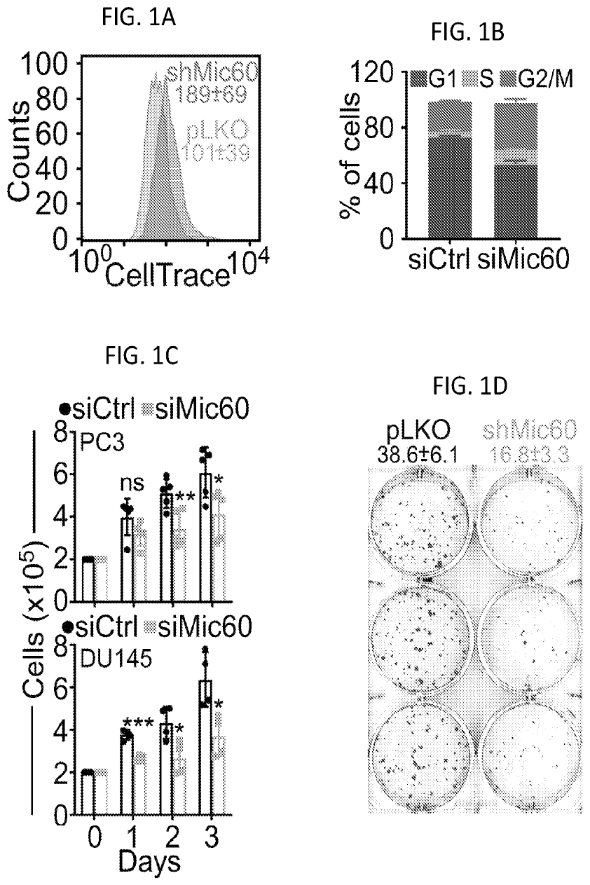

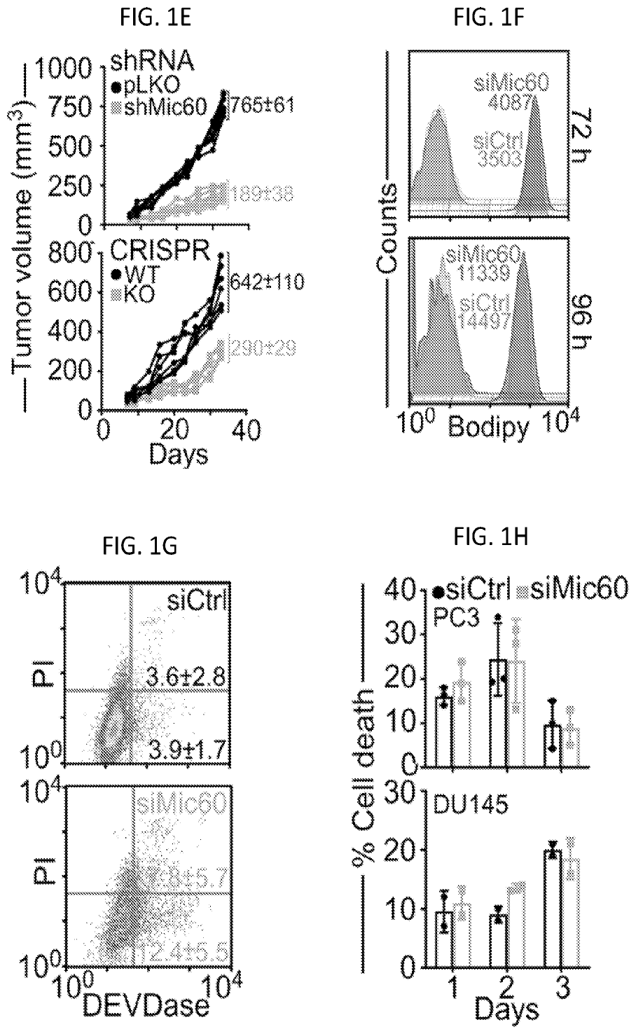

Based on these results, we next asked if the loss of mitochondrial fitness induced by Mic60 depletion affected tumor functions. Consistent with DNA damage, Mic60-depleted cells exhibited slower cell cycle progression (FIG. 1A) and accumulation of cells with G2/M DNA content throughout a 7-d culture (FIG. 1B). This resulted in a reduced proliferation of normal and tumor cell types (FIG. 1C), as well as an inhibition of colony formation (FIG. 1D). An analysis of the DepMap Portal revealed dependency scores <1 for all tumor types examined after Mic60 silencing by RNA interference (RNAi) or CRISPR-Cas9, consistent with a general requirement of Mic60 for tumor cell proliferation. Accordingly, PC3 clones with reduced Mic60 levels by shRNA or CRISPR-Cas9 formed slow-growing superficial tumors in immunocompromised mice (FIG. 1E) with decreased Ki-67 reactivity, a marker of cell proliferation. Conversely, control PC3 cells formed exponentially growing flank tumors (FIG. 1E) with high Ki-67 staining.

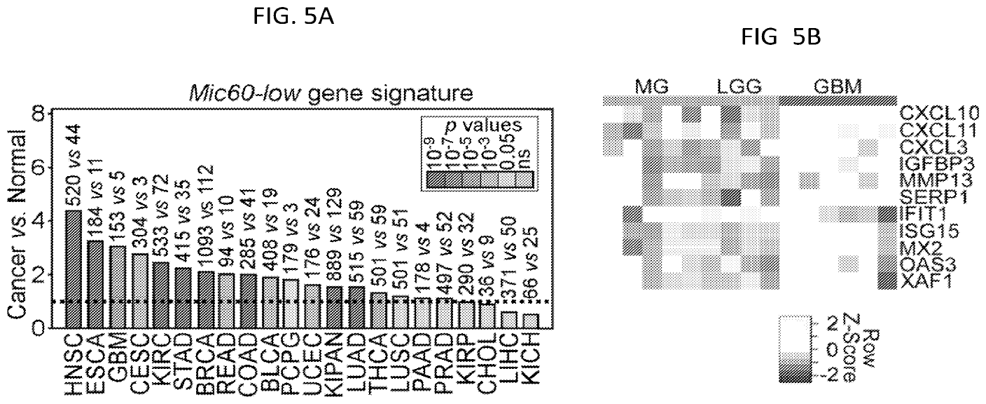

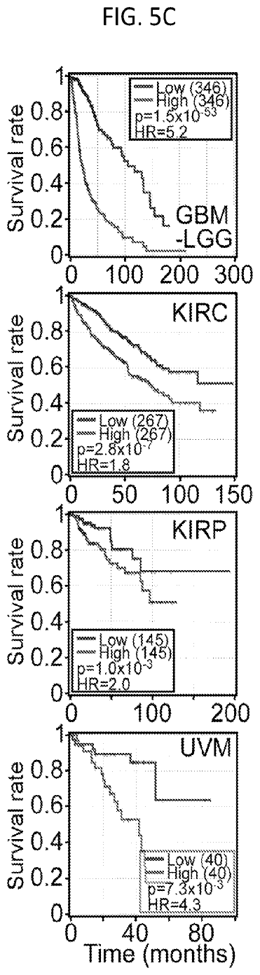

Despite cellular damage and activation of mitophagy, tumor cells with reduced Mic60 did not upregulate ferroptosis-associated genes, a type of cell death induced by mitochondrial stress, and oxidized lipid content, a marker of ferroptosis, was unchanged compared to control cultures (FIG. 1F). Similarly, Mic60 depletion only modestly affected mitochondrial apoptosis, as quantified by caspase (DEVDase) activity (FIG. 1G) or hypodiploid DNA content and flow cytometry. Finally, no significant differences in necroptotic cell death were observed in control or Mic60-silenced cells over a 3-d (FIG. 1H) or 5-d culture, by analysis of plasma membrane integrity and light microscopy.

### Mic60 Depletion Induces a Unique Innate Immunity and Cytokine Chemokine Gene Signature

Next, we looked at potential mechanisms of cellular adaptation in Mic60-low tumors. By RNA sequencing (RNA-Seq), Mic60 silencing induced unique transcriptional changes in PC3 cells with upregulation of a type I interferon (IFN) response and cytokines/chemokines reminiscent of a senescence-associated secretory phenotype (SASP) (FIG. 2A). The Mic60 transcriptome activated in these settings also comprised PI3K/Akt signaling (see below) as well as pathways of genomic integrity (NER), endoplasmic reticulum stress, unfolded protein response (UPR), pattern recognition (RIG-1), and cytoskeletal (ARP-WASP) remodeling (FIG. 2B). In contrast, Mic60 depletion suppressed eIF2a signaling (FIG. 2B).

In validation experiments, Mic60-depleted cells upregulated SASP-like cytokines (IL6, IL8, IL18, and IL1α), chemokines (CXCL2, CXCL3), protease (MMP13), and growth factor modulators (IGFBP7, IGFBP3), as well as effectors of IFN signaling, IFIT1, IFIT3, MX1, OAS1, ISG15, and IFITM1 by RT-PCR. IL23 and MMPI were not affected. Similar results were obtained at the protein level, as Mic60 silencing increased the expression of MX2 and MMP13 by Western blotting and heightened the release of cytokines (IL6, IL8, CXCL10), protease (MMP13), and IFNs (IFNα and IFNβ) in the cell supernatant compared to control transfectants. Finally, TCGA analysis demonstrated that all 52 IFN/SASP-like genes of the Mic60 transcriptome were significantly upregulated in Mic60-low tumors of the head and neck, brain (GBM), colon, rectum, kidney, and thyroid compared to normal tissues (FIG. 2C).

Mechanistically, siRNA silencing of STING, a key regulator of mitochondrion-directed innate immunity, abolished the increase in cytokine mRNA levels after Mic60 loss (FIG. 2D). In addition, Mic60 depletion was accompanied by an increased phosphorylation of STAT1 (Ser727) and extracellular release of HMGB1, which are two effectors of IFN signaling. In contrast, senescence-associated β-galactosidase staining was unchanged in control or Mic60-depleted cultures.

### Mic60 Regulation of Tumor Cell Invasion and Metastasis

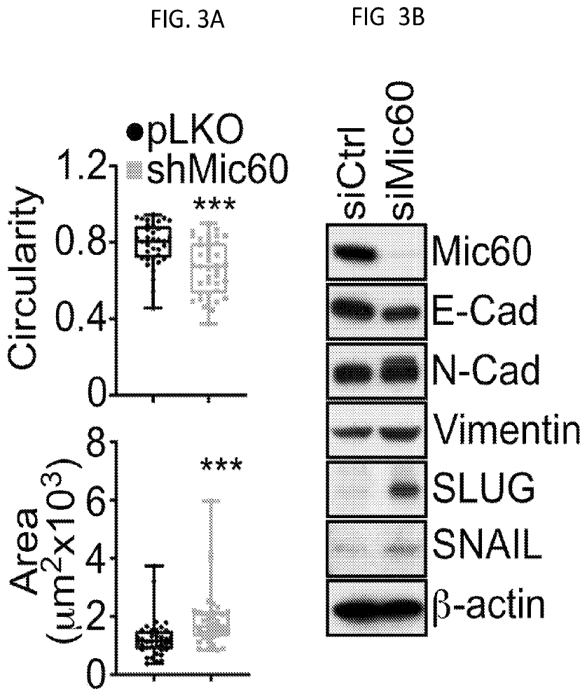

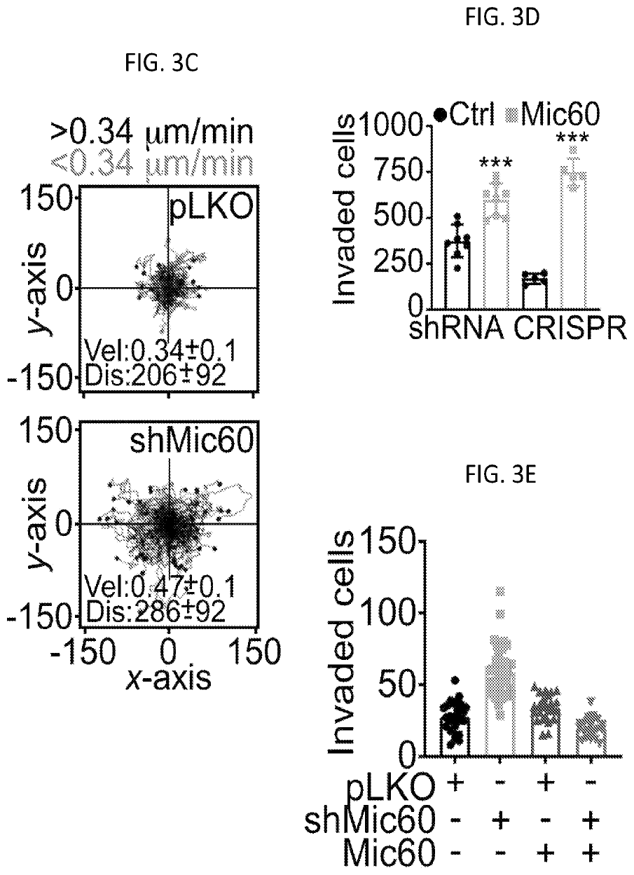

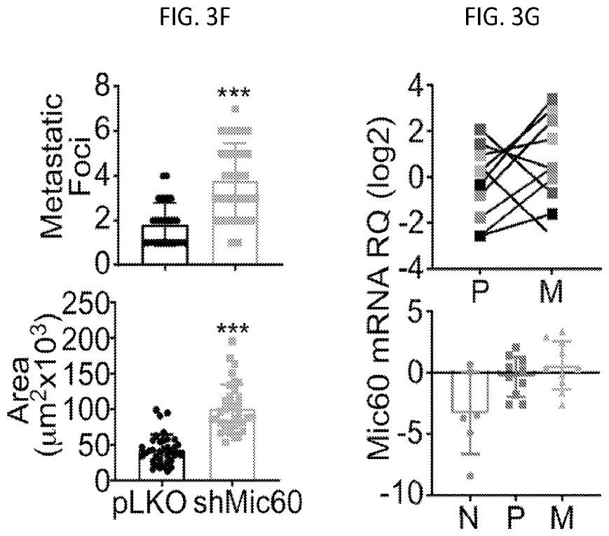

SASP signaling has been associated with increased tumor cell invasion and metastasis. Accordingly, Mic60 depletion changed the morphology of PC3 cells to a flattened, elongated, and spindle-shaped appearance, characterized by rearrangement of the actin cytoskeleton and redistribution of mitochondria to the cortical cytoskeleton. This was associated with reduced cellular roundness, increased surface area (FIG. 3A), and the appearance of epithelial-mesenchymal transition (EMT) markers, including E- and N-cadherin switch, and upregulation of vimentin, SLUG, and SNAIL (FIG. 3B). Functionally, Mic60 depletion enhanced FA turnover, increasing the fraction of new and decayed FA, while reducing stable FA. This resulted in a greater speed of single-cell movements, longer distance traveled by individual cells (FIG. 3C), and accelerated directional cell motility in a “wound” closure assay, shortening the half-time (t½) of wound closure from 16.3 h to 6.3 h. In addition, silencing of Mic60 increased tumor cell invasion across Matrigel (FIG. 3D), whereas re-expression of Mic60 complementary DNA (cDNA) in these settings normalized Matrigel invasion (FIG. 3E). As for signaling requirements, Mic60 silencing increased the phosphorylation of Focal Adhesion Kinase (FAK), and FAK targeting by siRNA or a small-molecule inhibitor (FAKᵢ) normalized FA dynamics and restored the single-cell motility of PC3 or LN229 cells to levels of control transfectants.

Although impaired in primary tumor growth (FIG. 1E), superficial flank tumors of Mic60-depleted PC3 cells gave rise to increased metastatic dissemination to the lungs of immunocompromised mice. As a second, independent model of metastasis, we injected control or Mic60-depleted PC3 cells in the spleen of immunocompromised animals and looked at liver metastasis after 11 d. Here, Mic60-silenced PC3 cells generated more numerous and larger liver metastases compared to controls (FIG. 3F). Based on these data, we next looked at matched patient samples of primary and metastatic LUAD. In this comparison, Mic60 mRNA levels increased in metastases compared to the primary tumor (FIG. 3G, Top) and the nonneoplastic lung tissue (FIG. 3G, Bottom), suggesting that Mic60 becomes re-expressed at established metastatic sites to support tumor cell proliferation.

### Regulation of Mitochondrial Dynamics and Cell Movements by Mic60

The mechanism(s) underlying increased tumor cell motility after Mic60 targeting were next investigated. Despite the loss of mitochondrial fitness, Mic60 depletion stimulated mitochondrial dynamics in LN229 cells and less consistently in other tumor types, resulting in higher rates of mitochondrial fission. Mitochondrial fusion was less affected. As a result, Mic60-depleted cells exhibited heightened subcellular mitochondrial trafficking, with a longer distance traveled by individual mitochondria and increased accumulation of mitochondria at the cortical cytoskeleton of LN229 and PC3 cells compared to controls.

Mechanistically, siRNA silencing of mitochondrial GTPase RHOT1 or RHOT2, which mediate mitochondrial trafficking in tumors, normalized the speed of mitochondrial movements and the distance traveled by individual mitochondria after Mic60 depletion. Buffering oxidative stress gave similar results, as the reconstitution of Mic60-silenced cells with antioxidant Prx3 corrected the increase in single-cell motility, lowered the speed of cell movements and the total distance traveled by individual cells to levels of control cultures, and reversed the increase in Matrigel invasion in these settings. Reconstitution of Mic60-silenced cells with a mitochondrial-targeted superoxide scavenger, MitoTempo, also normalized tumor cell invasion to control levels.

### Adaptive GCN2-Akt Signaling as Therapeutic Vulnerability in Mic60-Low Tumors

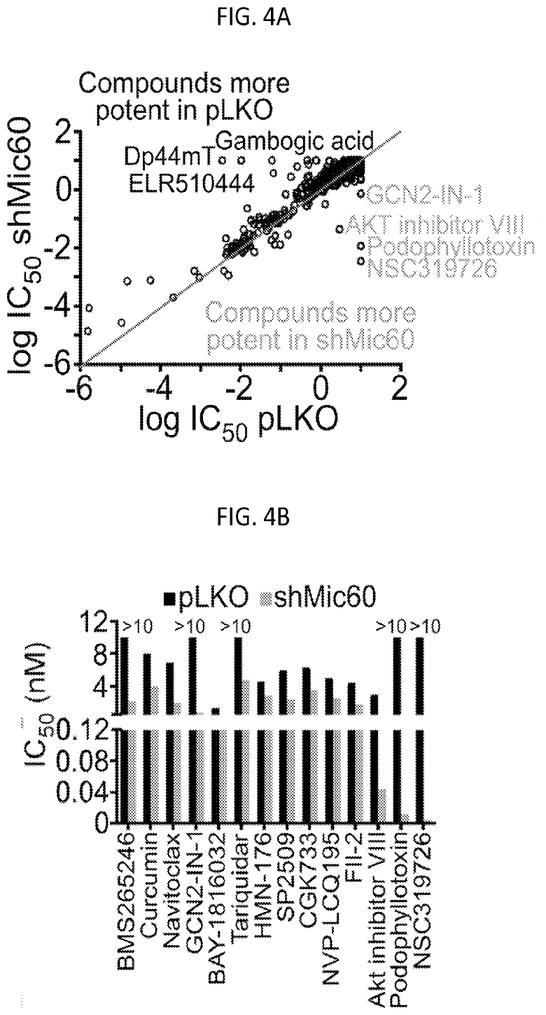

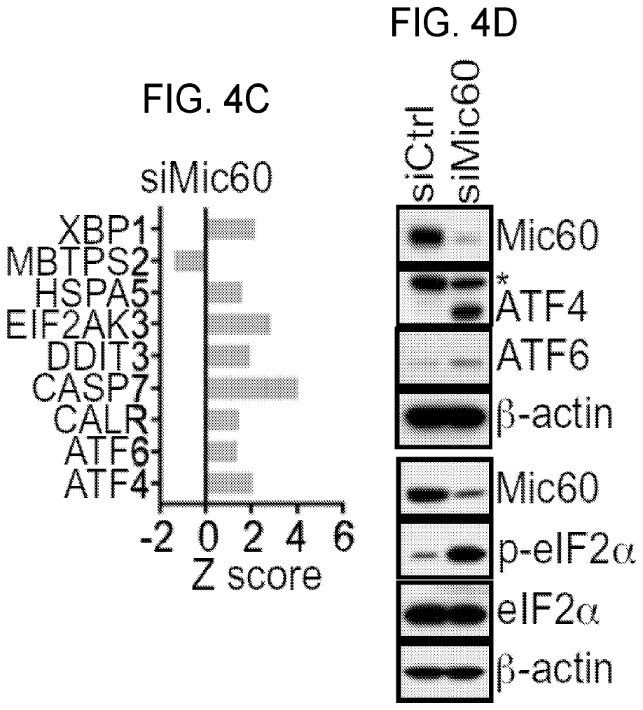

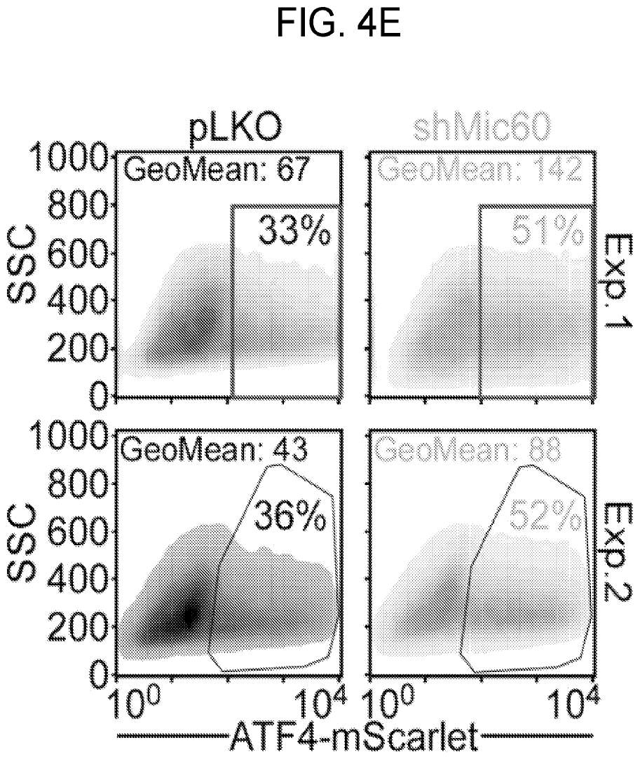

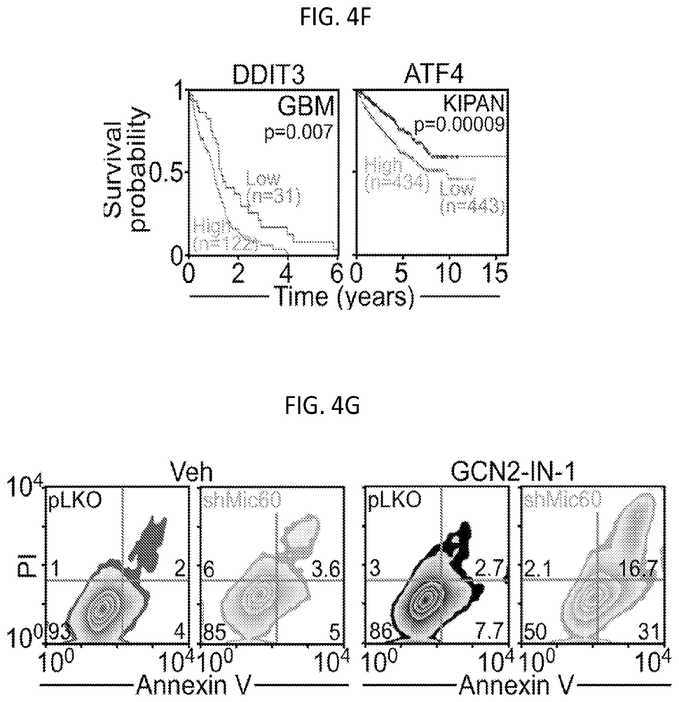

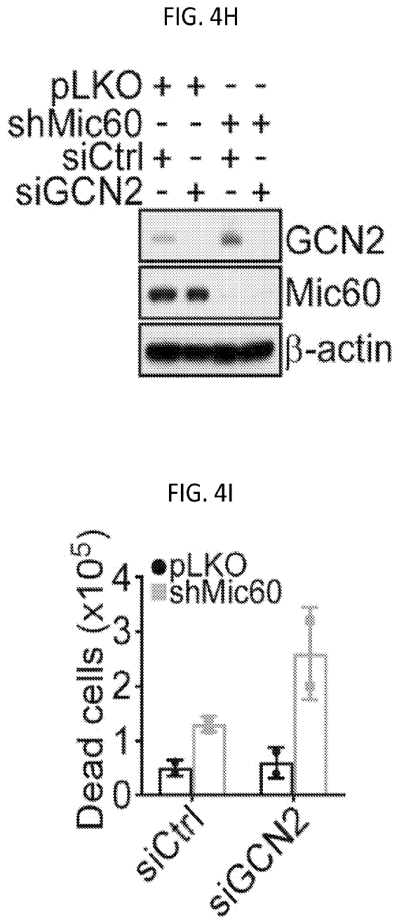

Finally, we asked if adaptive mechanisms activated in Mic60-low tumors exposed actionable therapeutic vulnerabilities. In a small-molecule drug screen, antagonists of Akt (Akt inhibitor VIII) or General Control Nonderepressible 2 kinase GCN2 (GCN2-IN-1) killed Mic60-silenced PC3 cells more efficiently than control cultures (FIG. 4A and FIG. 4B). Natural compounds, podophyllotoxin and curcumin, p53 reactivator NSC319726, Bcl2 pathway inhibitor, Navitoclax, P glycoprotein antagonist, Tariquidar, multi-CDK inhibitor, NVP-LCQ195 and histone lysine-specific demethylase-1 inhibitor, and SP2509 also showed preferential killing against Mic60-low PC3 cells (FIG. 4B). Consistent with these data, Mic60 silencing increased Akt phosphorylation, with downstream activation of Akt targets PDK1, BAD, p70S6K, and p27 quantified in a phosphoarray screen. Functionally, treatment with Akt inhibitor VIII or another small molecule Akt antagonist, MK2206, suppressed proliferation selectively of Mic60-depleted PC3 cells compared to control transfectants.

GCN2 is a critical eIF2α kinase in the integrated stress response (ISR). Accordingly, ISR effectors ATF4, ATF6, eIF2AK3, and calreticulin were selectively upregulated after Mic60 depletion by RNA-Seq analysis (FIG. 4C). This was accompanied by an increased expression of ATF4 and ATF6 by Western blotting (FIG. 4D) and translocation of transcriptionally active ATF4 to the nucleus of Mic60-low PC3 cells compared to parental cultures (FIG. 4E). Consistent with these data, Mic60 silencing induced a strong phosphorylation of eIF2α in PC3 cells, whereas total eIF2α was unaffected (FIG. 4D). Bioinformatics analysis of the Ivy Glioblastoma Atlas Project (available online at glioblastoma.alleninstitute.org) demonstrated that ISR regulators EIF2AK2, EIF2A, ATF4, XBP1, and DDIT3 were differentially increased in Mic60-low GBM and spatially localized within “pseudopalisades,” which are hypoxic hypercellular structures associated with greater invasiveness. Furthermore, high levels of ISR effectors DDIT3 and ATF4 correlated with shortened patient survival in Mic60-low GBM and kidney cancer (FIG. 4F) but not Mic60-high BRCA and LUAD. Functionally, a small-molecule GCN2 inhibitor (GCN2-IN-1) inhibited proliferation (pLKO, 3.3×10⁵±0.62×10⁵ cells; shMic60, 1.95×10⁵±0.4×10⁵ cells; P=0.009) and activated Annexin V-associated apoptosis (FIG. 4G) and caspase-dependent cell death (pLKO, 8.4±6.3%; shMic60, 23.1±5.6%; P=0.04) preferentially in Mic60-silenced PC3 cells compared to control cultures. As an independent approach, siRNA silencing of GCN2 also increased cell death selectively in Mic60-silenced cultures (FIG. 4H and FIG. 4I).

In this study, we have shown that Mic60, an essential scaffold of mitochondrial structure, is heterogeneously expressed and often reduced in human cancer compared to normal tissues. As modeled in tumor cell lines, even a partial reduction in Mic60 levels was sufficient to induce an acute loss of mitochondrial fitness, leading to bioenergetics defects, cellular starvation, and oxidative stress. Despite the activation of quality-control measures of autophagy and mitophagy, tumor cells harboring such extensively damaged, ghost mitochondria managed to evade cell death, slowed down cell proliferation, and activated mitochondrial dynamics to fuel increased cell invasion and metastasis. This response was accompanied by the expression of an IFN/SASP-like transcriptome, as well as adaptive activation of GCN2/Akt survival signaling, which provided an actionable therapeutic target in these metastasis-prone tumors.

The basis for the heterogeneous expression of Mic60 in cancer remains to be elucidated. This may result from mitochondrial and/or environmental stress conditions associated with tumor growth, including defective oxidative phosphorylation, or alternatively, mechanisms of tumor evolution, as suggested here by Mic60 downregulation during differentiation of patient-derived GBM neurospheres.

Irrespectively, decreased Mic60 levels generate subpar, ghost mitochondria that bear striking similarities to the mitochondrial defects seen in aging. Consistent with this, parallel, in vivo models of aging have been associated with reduced Mic60 levels, heightened SASP signaling, mitochondria-to-nuclei retrograde gene expression, and a general, proinflammatory environment. However, key differences between aged and Mic60-low mitochondria have also been noted. Aside from distinct profiles of cytokine induction, Mic60-depleted tumor cells were negative for senescence-associated β-galactosidase and did not undergo permanent G1 cell cycle arrest, and gene expression changes in these settings were unrelated to p53 status.

A unique adaptive response of Mic60-low tumors was the upregulation of a nuclear transcriptome combining a type I IFN response characteristic of innate immunity and cytokines/chemokines reminiscent of SASP signaling. Mechanistically, mitochondrial stressors induced by Mic60 loss, such as ROS, loss of membrane integrity, and energy starvation, have all been associated with mitochondria-to-nuclei retrograde gene expression. Activation of a type I IFN response in these settings fits well with a key role of mitochondria in innate immunity, in line with the activation of STAT1 and the requirement of STING for cytokine production observed here. While there is evidence that SASP-associated cytokine/chemokine signaling promotes tumor growth, favors an immunosuppressive microenvironment, and enhances metastasis, the role of a type I IFN response in cancer is likely time- and context-specific. Whereas acute IFN signaling has been associated with antitumor immunity and improved treatment responses, sustained inflammatory conditions are protumorigenic and chronic IFN stimulation enables myeloid-directed immunosuppression and propagation of cancer stemness.

Consistent with this scenario, Mic60-low tumors switched from a proliferative to a highly motile and prometastatic phenotype, contributed by EMT, sustained FAK phosphorylation, and heightened mitochondrial dynamics. Described as phenotype switching, this reversible transition between proliferative and migratory states has been proposed as a potential escape mechanism for tumor cells to leave a stress-laden, unfavorable microenvironment and colonize distant sites, i.e., metastasis. Regulators of mitochondrial dynamics, such as syntaphilin, FUNDC1, and now Mic60 depletion, are important mediators of phenotype switching, reprogramming oxidative bioenergetics, and redox balance to promote heightened cell migration and invasion at the expense of cell proliferation. Consistent with this model, oxidative stress generated in Mic60-low tumors was a key mediator of increased mitochondrial trafficking and tumor cell movements, in keeping with a central role of ROS in tumor cell motility, EMT, and metastasis.

Despite an extensive loss of mitochondrial fitness, activation of autophagy/mitophagy, and high ROS production, Mic60-low tumor cells managed to persist likely through the activation of compensatory cell survival mechanisms. The induction of GCN2/ISR as well as Akt signaling observed in these settings appears ideally poised to adjust metabolism under stress, preserve mitochondrial integrity and oppose cell death. Although correlating with shortened patient survival, a potential dependence of Mic60-low tumors to GCN2/ISR/Akt adaptive signaling was therapeutically exploitable, and the studies provided here demonstrated that pharmacologic or genetic targeting of this pathway can restore mitochondrial cell death and inhibit proliferation selectively of Mic60-low tumor cells.

In summary, we have shown that persistent, acutely degraded ghost mitochondria are major signaling hubs in cancer, driving multiple, adaptive responses of nuclear gene expression, ISR activation, and suppression of mitochondrial cell death to enable metastatic competence. This reinforces the role of mitochondrial reprogramming as an important therapeutic target in cancer, especially in hard-to-treat and metastasis-prone malignancies with currently limited therapeutic options.

### Example 3: Mitochondrial Fitness and Cancer Risk

Recent studies have shown that tumors with reduced expression of the mitochondrial structural protein, Mic60 upregulate a unique transcriptome comprising regulators of IFN signaling and SASP. In a first set of experiments, we narrowed the a Mic60-low transcriptome described in Example 2 to 11 genes representative of IFN response (IFN Short: IFIT1, ISG15, MX2, OAS3, XAF1) and SASP signaling (SASP Short: CXCL10, CXCL11, CXCL3, MMP13, IGFBP3, SERPINE1) based on >5-fold upregulation and role in cancer. In analysis of TCGA datasets, the 11-gene Mic60-low gene signature was differentially expressed in multiple, genetically unrelated tumor types (p<0.05), compared to the corresponding normal tissues (FIG. 5A). The highly heterogeneous distribution of Mic60 in PDAC may explain why expression of the Mic60-low gene signature did not reach statistical significance in this tumor type, compared to normal tissue. In validation studies using GBM as a tumor model (FIG. 5A), the 11-gene Mic60-low gene signature was highly expressed in primary, patient-derived GBM tissue samples, compared to LGG and normal brain parenchyma, i.e., tumor-free margins (MG) (FIG. 5B). Examination of the Ivy Glioblastoma Atlas Project dataset (https://glioblastoma.alleninstitute.org) revealed that representative genes in the Mic60-low gene signature, SERPINE1, IL8, IL6, IL1α and IGFBP3 spatially segregated within garland-like hypoxic hypercellular structures known as GBM pseudopalisades and associated with high invasiveness. Consistent with more aggressive phenotype, high expression of the Mic60-low gene signature was associated with dramatically reduced overall patient survival in the GBM-LGG dataset of TCGA (p=1.5×10⁻⁵³, HR=5.2) (FIG. 5D). Similar results were observed in TCGA datasets of other tumor types, where increased expression of the Mic60-low gene signature was associated with shorted patient survival in kidney cancer (both KIRC and KIRP), uveal melanoma (UVM) (FIG. 5C), testicular germ cell tumors (HR>4, p=0.0074, N=139), and thymomas (HR=3.1, p=0.036, N=119).

Next, we focused on PDAC as a second tumor model to evaluate the Mic60 pathway in cancer. Consistent with recent observations, analysis of patient-derived tissue samples by immunohistochemistry demonstrated that Mic60 expression was highly heterogeneous in PDAC, with moderate expression in pancreatic intraepithelial neoplasia and well-differentiated PDAC to undetectable levels in high-grade, basaloid PDAC.

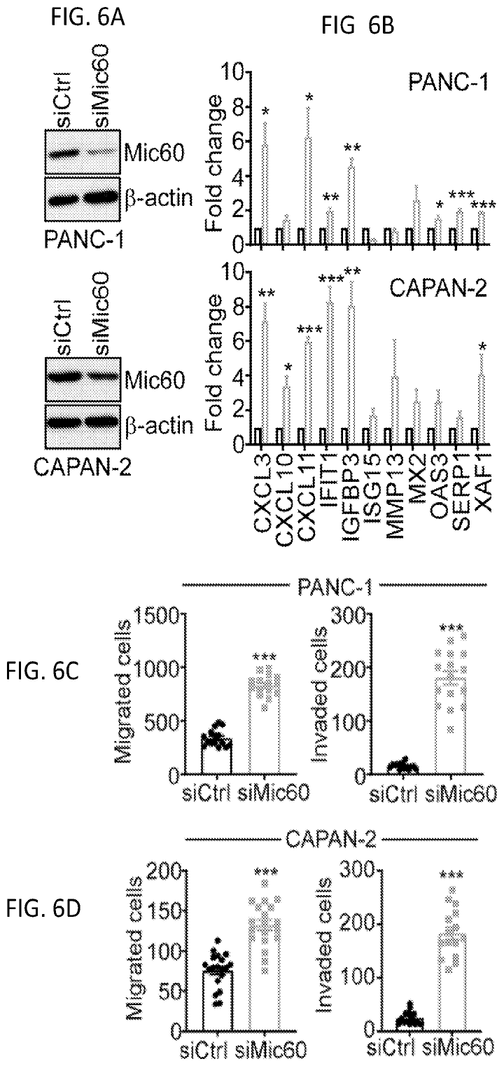

To mimic the effect of reduced Mic60 levels in PDAC, we next silenced endogenous Mic60 expression in PDAC cell lines, PANC-1 and CAPAN-2 by siRNA (FIG. 6A). Consistent with previous observations, Mic60 silencing was associated with increased expression of the Mic60-low gene signature in PDAC cell lines, albeit with cell type-specific differences (FIG. 6B). Functionally, Mic60 silencing was associated with dramatically increased PANC-1 (FIG. 6C) and CAPAN-2 (FIG. 6D) cell migration and invasion.

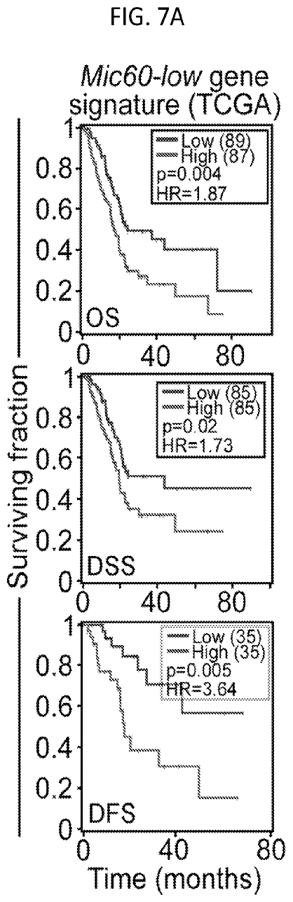

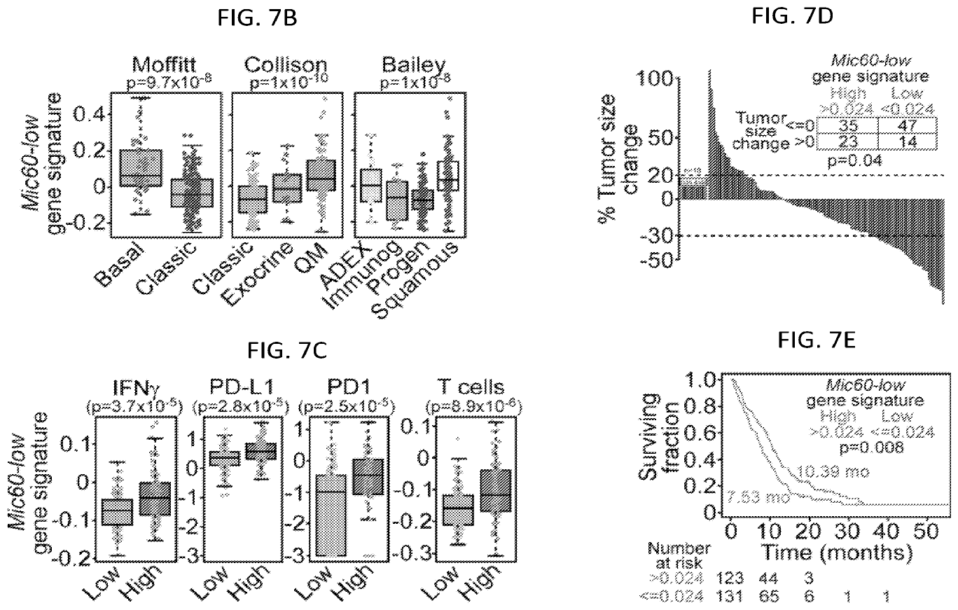

Based on these data, we next assessed the impact of the Mic60-low gene signature on PDAC risk. First, expression of the Mic60-low gene signature in the TCGA dataset of PDAC was associated with shortened overall survival (HR=1.87, p=0.004, N=176), disease-specific survival (HR=1.73, p=0.02, N=170) and disease-free status (HR=3.6, p=0.005, N=70), independently of age, gender, or stage (FIG. 7A). Next, we examined RNA-Seq data from patients (N=195) enrolled in the COMPASS trial. In this patient cohort, increased expression of the Mic60-low gene signature was associated with aggressive molecular variants of basal, quasi-mesenchymal and squamous PDAC (FIG. 7B), inflammation-associated expression of IFNγ (p=4×10⁻⁵), PD1 (p=3×10⁻⁵), PD-L1 (p=3×10⁻⁵) and T cells (p=9×106) (FIG. 7C), FOLFIRINOX failure (FIG. 7D) and shorter overall survival (FIG. 7E). Conversely, the Mic60-low gene signature did not correlate with hypoxia status (p=0.7, Wilcoxon rank sum test), cell cycle progression (p=0.46, Wilcoxon rank sum test) or four genomic subtypes of PDAC, including stable, locally rearranged, scattered and unstable (p=0.39, Kruskal-Wallis test).

As a third dataset, we examined the expression of the Mic60-low gene signature in the CPTAC dataset. In this cohort, higher values of the Mic60-low gene signature independently correlated with differential expression in PDAC vs. normal tissue (p=0.006) and basal vs. classical molecular tumor variants (p=0.005).

The 11 gene Mic60-low gene signature is the first gene signature of mitochondrial reprogramming in cancer linked to aggressive disease subtypes, treatment failure and abbreviated patient survival. Despite progress in molecular and genomic profiling, the role of mitochondrial reprogramming in PDAC is only beginning to emerge and metabolic biomarkers for detection and treatment in these settings remain urgently needed. Mic60 is an essential structural protein in mitochondria and its reduced expression triggers acute organelle dysfunction, loss of bioenergetics and oxidative stress. The basis for the reduced and often undetectable levels of Mic60 in many human tumors remains to be elucidated. However, it is likely that the unique transcriptome of SASP and IFN signaling upregulated in these Mic60-low tumors represents an adaptive response to loss of mitochondrial fitness that confers increased metastatic ability. Accordingly, SASP signaling has been linked to increased tumor cell invasion and metastasis, whereas chronic activation of a type I IFN response enhances pro-tumorigenic inflammation and local immunosuppression.

Based on the findings presented here, the Mic60-low gene signature can be utilized in an easily accessible, point-of-service molecular tool to stratify patient risk in PDAC and potentially other malignancies, including GBM.

All publications cited in this specification are incorporated herein by reference. U.S. Provisional Patent Application No. 63/270,883, filed Oct. 22, 2021, and U.S. Provisional Patent Application No. 63/371,600, filed Aug. 16, 2022, are incorporated herein by reference. While the invention has been described with reference to particular embodiments, it will be appreciated that modifications can be made without departing from the spirit of the invention. Such modifications are intended to fall within the scope of the appended claims.

1. A method of treating cancer in a subject in need thereof, the method comprising:
a) obtaining a tumor sample from the subject;
b) detecting expression levels of genes in the tumor sample, wherein the genes comprise
MMP13, XAF1, IGFBP3, MX2, IFIT1, ISG15, OAS3, CXCL10, SERPINE1, CXCL11, and CXCL3,
wherein a change in the expression level(s) of the one or more genes as compared to a control level indicates Mic60-depleted cancer; and
c) treating the subject for the Mic60-depleted cancer.

2. A method of treating cancer in a subject in need thereof, the method comprising:
having obtained information about expression levels of genes in a tumor sample obtained from the subject, wherein the genes comprise
MMP13, XAF1, IGFBP3, MX2, IFIT1, ISG15, OAS3, CXCL10, SERPINE1, CXCL11, and CXCL3, wherein the expression level(s) of one more of the genes as compared to a control level indicates Mic60-depleted cancer, administering a treatment for the cancer to the subject.

3. A method of reducing, inhibiting, and/or preventing tumor metastasis in a subject in need thereof, the method comprising:
having obtained information about expression levels of genes in a tumor sample obtained from the subject, wherein the genes comprise
MMP13, XAF1, IGFBP3, MX2, IFIT1, ISG15, OAS3, CXCL10, SERPINE1, CXCL11, CXCL3, wherein the expression level(s) of the one or more genes as compared to a control level indicates Mic60-depleted cancer, administering a treatment for the cancer to the subject.

4. The method according to claim 1, wherein the genes further comprise one or more of IFIT3, IFITM1, IFIT1, OAS1, IFI35, MX1, ISG15, IFI6, IFITM2, TAP1, STAT1, IFITM3, STAT2, PSMB8, IRF1, IFNB1, IRF9, IFNGR1, RELA, BAK1, XAF1, IGFBP3, MX2, IFIT1, IFIT2, CXCL10, SERPIN1, OAS2, MX1, ISG15, OAS3, IL6, IL8, MMP13, CXCL3, CXCL2, CCL26, CCL20, VEGFC, IL15, CXCL6, CXCL1, SERPRINB2, ATF5, CD55, KITLG, IGFBP6, CD276, LAMB2, ANG, ETS2, INHBA, AXL, AREG, RELA, and MIF.

5. The method according to claim 1, wherein the genes consist of XAF1, IGFBP3, MX2, IFIT1, ISG15, OAS3, CXCL10, SERPINE1, CXCL11, CXCL3, and MMP13.

6. The method according to claim 1, wherein the expression level of one or more of the genes is determined by detecting an RNA level, optionally by qPCR.

7. The method according to claim 1, wherein the expression level of one or more of the genes is determined detecting a gene product/protein level, optionally by immunohistochemistry.

8. The method according to claim 1, wherein the gene expression levels for the tumor sample are compared those of a control, wherein the control is a normal tissue sample.

9. The method according to claim 1, wherein the cancer is head and neck, esophageal, glioblastoma (GBM), kidney (clear cell and papillary cell carcinoma), colorectal, glioma, pancreatic ductal adenocarcinoma (PDAC), uveal melanoma, adrenocortical carcinoma, breast, neuroendocrine system, carcinoid, cervical, liver, lung, lymphoma, melanoma, ovarian, pancreatic, prostate, skin, stomach, testis, thyroid, or urothelial.

10. The method according to claim 1, wherein the treatment comprises administering a focal adhesion kinase (FAK) inhibitor to the subject.

11. The method according to claim 10, wherein the FAK inhibitor is a small molecule inhibitor, shRNA, or siRNA.

12. (canceled)

13. The method according to claim 1, wherein the treatment comprises administering an GCN2 inhibitor and/or Akt inhibitor to the subject.

14. The method according to claim 13, wherein the GCN2 inhibitor or the Akt inhibitor is a small molecule inhibitor, shRNA, or siRNA.

17.-17. (canceled)

18. The method according to claim 1, wherein the treatment comprises administering a RHOT1 inhibitor and/or RHOT2 inhibitor to the subject.

19. The method according to claim 18, wherein the RHOT1 inhibitor and/or RHOT2 inhibitor is a small molecule inhibitor, shRNA, or siRNA.

20. The method according to claim 1, wherein the treatment comprises administering podophyllotoxin, curcumin, NSC319726, Navitoclax, Tariquidar, NVP-LCQ195, SP2509, BMS265246, GCN2-IN-1, BAY-1816032, CGK733, FII-2, and/or Akt inhibitor VIII.

21. The method according to claim 1, wherein the treatment comprises administering a chemotherapy, radiation therapy, immunotherapy, hormone therapy, stem cell or bone marrow transplant, and/or surgical resection.

22. The method according to claim 1, wherein the treatment comprises FOLFIRINOX or FOLFOXIRI.

23. A composition comprising a collection of probes, primers, and/or antibodies suitable for detection of the expression and/or expression levels of a collection of genes, or their products, in a tumor sample, wherein the collection of genes comprises at least eleven of
Mic60, XAF1, IGFBP3, MX2, IFIT1, ISG15, OAS3, CXCL10, SERPINE1, CXCL11, CXCL3, MMP13, IFIT3, IFITM1, IFIT1, OAS1, IFI35, MX1, ISG15, IFI6, IFITM2, TAP1, STAT1, IFITM3, STAT2, PSMB8, IRF1, IFNB1, IRF9, IFNGR1, RELA, BAK1, IFNGR2, MED14, IL18, IL1α, IGFBP7, IFIT2, OAS2, MX1, IL6, IL8, CXCL2, CCL26, CCL20, VEGFC, IL15, CXCL6, CXCL1, SERPINB2, ATF5, CD55, KITLG, IGFBP6, CD276, LAMB2, ANG, ETS2, INHBA, AXL, AREG, RELA, and/or MIF.

24. The composition according to claim 23, wherein the collection of genes consists of XAF1, IGFBP3, MX2, IFIT1, ISG15, OAS3, CXCL10, SERPINE1, CXCL11, CXCL3, and MMP13.


SEQUENCE LISTING (11 sequences)

SEQ ID NO: 1 - DNA, 1416 bp, Homo sapiens
  source 1..1416: mol_type = other DNA
atgcatccaggggtcctggctgccttcctcttcttgagctggactcattgtcgggccctgccccttcccagtggtggtgatgaagatgatttgtctgaggaagacctccagtttgcagagcgctacctgagatcatactaccatcctacaaatctcgcgggaatcctgaaggagaatgcagcaagctccatgactgagaggctccgagaaatgcagtctttcttcggcttagaggtgactggcaaacttgacgataacaccttagatgtcatgaaaaagccaagatgcggggttcctgatgtgggtgaatacaatgttttccctcgaactcttaaatggtccaaaatgaatttaacctacagaattgtgaattacacccctgatatgactcattctgaagtcgaaaaggcattcaaaaaagccttcaaagtttggtccgatgtaactcctctgaattttaccagacttcacgatggcattgctgacatcatgatctcttttggaattaaggagcatggcgacttctacccatttgatgggccctctggcctgctggctcatgcttttcctcctgggccaaattatggaggagatgcccattttgatgatgatgaaacctggacaagtagttccaaaggctacaacttgtttcttgttgctgcgcatgagttcggccactccttaggtcttgaccactccaaggaccctggagcactcatgtttcctatctacacctacaccggcaaaagccactttatgcttcctgatgacgatgtacaagggatccagtctctctatggtccaggagatgaagaccccaaccctaaacatccaaaaacgccagacaaatgtgacccttccttatcccttgatgccattaccagtctccgaggagaaacaatgatctttaaagacagattcttctggcgcctgcatcctcagcaggttgatgcggagctgtttttaacgaaatcattttggccagaacttcccaaccgtattgatgctgcatatgagcacccttctcatgacctcatcttcatcttcagaggtagaaaattttgggctcttaatggttatgacattctggaaggttatcccaaaaaaatatctgaactgggtcttccaaaagaagttaagaagataagtgcagctgttcactttgaggatacaggcaagactctcctgttctcaggaaaccaggtctggagatatgatgatactaaccatattatggataaagactatccgagactaatagaagaagacttcccaggaattggtgataaagtagatgctgtctatgagaaaaatggttatatctattttttcaacggacccatacagtttgaatacagcatctggagtaaccgtattgttcgcgtcatgccagcaaattccattttgtggtgttaa

SEQ ID NO: 2 - DNA, 519 bp, Homo sapiens
  source 1..519: mol_type = other DNA
atgccggcagcgcaggaaagtcaagaccaggcaggggaaagaatttcagctcctgaaagggaaatctactgtcattattgcaaccaaatgattccagaaaataagtatttccaccatatgggtaaatgttgtccagactcagagtttaagaaacactttcctgttggaaatccagaaattcttccttcatctcttccaagtcaagctgctgaaaatcaaacttccacgatggagaaagatgttcgtccaaagacaagaagtataaacagatttcctcttcattctgaaagttcatcaaagaaagcaccaagaagcaaaaacaaaaccttggatccacttttgatgtcagagcccaagcccaggaccagctcccctagaggagataaagcagcctatgacattctgaggagatgttctcagtgtggcatcctgcttcccctgccgatcctaaatcaacatcaggagaaatgccggtggttagcttcatcaaaaggaaaacaagtgagaaatttcagctag

SEQ ID NO: 3 - DNA, 876 bp, Homo sapiens
  source 1..876: mol_type = other DNA
atgcagcgggcgcgacccacgctctgggccgctgcgctgactctgctggtgctgctccgcgggccgccggtggcgcgggctggcgcgagctcggcgggcttgggtcccgtggtgcgctgcgagccgtgcgacgcgcgtgcactggcccagtgcgcgcctccgcccgccgtgtgcgcggagctggtgcgcgagccgggctgcggctgctgcctgacgtgcgcactgagcgagggccagccgtgcggcatctacaccgagcgctgtggctccggccttcgctgccagccgtcgcccgacgaggcgcgaccgctgcaggcgctgctggacggccgcgggctctgcgtcaacgctagtgccgtcagccgcctgcgcgcctacctgctgccagcgccgccagctccaggaaatgctagtgagtcggaggaagaccgcagcgccggcagtgtggagagcccgtccgtctccagcacgcaccgggtgtctgatcccaagttccaccccctccattcaaagataatcatcatcaagaaagggcatgctaaagacagccagcgctacaaagttgactacgagtctcagagcacagatacccagaacttctcctccgagtccaagcgggagacagaatatggtccctgccgtagagaaatggaagacacactgaatcacctgaagttcctcaatgtgctgagtcccaggggtgtacacattcccaactgtgacaagaagggattttataagaaaaagcagtgtcgcccttccaaaggcaggaagcggggcttctgctggtgtgtggataagtatgggcagcctctcccaggctacaccaccaaggggaaggaggacgtgcactgctacagcatgcagagcaagtag

SEQ ID NO: 4 - DNA, 2148 bp, Homo sapiens
  source 1..2148: mol_type = other DNA
atgtctaaggcccacaagccttggccctaccggaggagaagtcaattttcttctcgaaaatacctgaaaaaagaaatgaattccttccagcaacagccaccgccattcggcacagtgccaccacaaatgatgtttcctccaaactggcagggggcagagaaggacgctgctttcctcgccaaggacttcaactttctcactttgaacaatcagccaccaccaggaaacaggagccaaccaagggcaatggggcccgagaacaacctgtacagccagtacgagcagaaggtgcgcccctgcattgacctcatcgactccctgcgggctctgggtgtggagcaggacctggccctgccagccatcgccgtcatcggggaccagagctcgggcaagagctctgtgctggaggcactgtcaggagtcgcgcttcccagaggcagcggaatcgtaaccaggtgtccgctggtgctgaaactgaaaaagcagccctgtgaggcatgggccggaaggatcagctaccggaacaccgagctagagcttcaggaccctggccaggtggagaaagagatacacaaagcccagaacgtcatggccgggaatggccggggcatcagccatgagctcatcagcctggagatcacctcccctgaggttccagacctgaccatcattgaccttcccggcatcaccagggtggctgtggacaaccagccccgagacatcggactgcagatcaaggctctcatcaagaagtacatccagaggcagcagacgatcaacttggtggtggttccctgtaacgtggacattgccaccacggaggcgctgagcatggcccatgaggtggacccggaaggggacaggaccatcggtatcctgaccaaaccagatctaatggacaggggcactgagaaaagcgtcatgaatgtggtgcggaacctcacgtaccccctcaagaagggctacatgattgtgaagtgccggggccagcaggagatcacaaacaggctgagcttggcagaggcaaccaagaaagaaattacattctttcaaacacatccatatttcagagttctcctggaggaggggtcagccacggttccccgactggcagaaagacttaccactgaactcatcatgcatatccaaaaatcgctcccgttgttagaaggacaaataagggagagccaccagaaggcgaccgaggagctgcggcgttgcggggctgacatccccagccaggaggccgacaagatgttctttctaattgagaaaatcaagatgtttaatcaggacatcgaaaagttagtagaaggagaagaagttgtaagggagaatgagacccgtttatacaacaaaatcagagaggattttaaaaactgggtaggcatacttgcaactaatacccaaaaagttaaaaatattatccacgaagaagttgaaaaatatgaaaagcagtatcgaggcaaggagcttctgggatttgtcaactacaagacatttgagatcatcgtgcatcagtacatccagcagctggtggagcccgcccttagcatgctccagaaagccatggaaattatccagcaagctttcattaacgtggccaaaaaacattttggcgaatttttcaaccttaaccaaactgttcagagcacgattgaagacataaaagtgaaacacacagcaaaggcagaaaacatgatccaacttcagttcagaatggagcagatggttttttgtcaagatcagatttacagtgttgttctgaagaaagtccgagaagagatttttaaccctctggggacgccttcacagaatatgaagttgaactctcattttcccagtaatgagtcttcggtttcctcctttactgaaataggcatccacctgaatgcctacttcttggaaaccagcaaacgtctcgccaaccagatcccatttataattcagtattttatgctccgagagaatggtgactccttgcagaaagccatgatgcagatactacaggaaaaaaatcgctattcctggctgcttcaagagcagagtgagaccgctaccaagagaagaatccttaaggagagaatttaccggctcactcaggcgcgacacgcactctgtcaattctccagcaaagagatccactga

SEQ ID NO: 5 - DNA, 1437 bp, Homo sapiens
  source 1..1437: mol_type = other DNA
atgtgtacaaatggtgatgatcatcaggtcaaggatagtctggagcaattgagatgtcactttacatgggagttatccattgatgacgatgaaatgcctgatttagaaaacagagtcttggatcagattgaattcctagacaccaaatacagtgtgggaatacacaacctactagcctatgtgaaacacctgaaaggccagaatgaggaagccctgaagagcttaaaagaagctgaaaacttaatgcaggaagaacatgacaaccaagcaaatgtgaggagtctggtgacctggggcaactttgcctggatgtattaccacatgggcagactggcagaagcccagacttacctggacaaggtggagaacatttgcaagaagctttcaaatcccttccgctatagaatggagtgtccagaaatagactgtgaggaaggatgggccttgctgaagtgtggaggaaaaaattatgaacgggccaaggcctgctttgaaaaggtgcttgaagtggaccctgaaaaccctgaatccagcgctgggtatgcgatctctgcctatcgcctggatggctttaaattagccacaaaaaatcacaagccattttctttgcttcccctaaggcaggctgtccgcttaaatccagacaatggatatattaaggttctccttgccctgaagcttcaggatgaaggacaggaagctgaaggagaaaagtacattgaagaagctctagccaacatgtcctcacagacctatgtctttcgatatgcagccaagttttaccgaagaaaaggctctgtggataaagctcttgagttattaaaaaaggccttgcaggaaacacccacttctgtcttactgcatcaccagatagggctttgctacaaggcacaaatgatccaaatcaaggaggctacaaaagggcagcctagagggcagaacagagaaaagctagacaaaatgataagatcagccatatttcattttgaatctgcagtggaaaaaaagcccacatttgaggtggctcatctagacctggcaagaatgtatatagaagcaggcaatcacagaaaagctgaagagaattttcaaaaattgttatgcatgaaaccagtggtagaagaaacaatgcaagacatacatttccactatggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctacaaattggaaggaaatatgaatgaagccctggagtactatgagcgggccctgagactggctgctgactttgagaactctgtgagacaaggtccttag

SEQ ID NO: 6 - DNA, 498 bp, Homo sapiens
  source 1..498: mol_type = other DNA
atgggctgggacctgacggtgaagatgctggcgggcaacgaattccaggtgtccctgagcagctccatgtcggtgtcagagctgaaggcgcagatcacccagaagatcggcgtgcacgccttccagcagcgtctggctgtccacccgagcggtgtggcgctgcaggacagggtcccccttgccagccagggcctgggccccggcagcacggtcctgctggtggtggacaaatgcgacgaacctctgagcatcctggtgaggaataacaagggccgcagcagcacctacgaggtacggctgacgcagaccgtggcccacctgaagcagcaagtgagcgggctggagggtgtgcaggacgacctgttctggctgaccttcgaggggaagcccctggaggaccagctcccgctgggggagtacggcctcaagcccctgagcaccgtgttcatgaatctgcgcctgcggggaggcggcacagagcctggcgggcggagctaa

SEQ ID NO: 7 - DNA, 3264 bp, Homo sapiens
  source 1..3264: mol_type = other DNA
atggacttgtacagcaccccggccgctgcgctggacaggttcgtggccagaaggctgcagccgcggaaggagttcgtagagaaggcgcggcgcgctctgggcgccctggccgctgccctgagggagcgcgggggccgcctcggtgctgctgccccgcgggtgctgaaaactgtcaagggaggctcctcgggccggggcacagctctcaagggtggctgtgattctgaacttgtcatcttcctcgactgcttcaagagctatgtggaccagagggcccgccgtgcagagatcctcagtgagatgcgggcatcgctggaatcctggtggcagaacccagtccctggtctgagactcacgtttcctgagcagagcgtgcctggggccctgcagttccgcctgacatccgtagatcttgaggactggatggatgttagcctggtgcctgccttcaatgtcctgggtcaggccggctccggcgtcaaacccaagccacaagtctactctaccctcctcaacagtggctgccaagggggcgagcatgcggcctgcttcacagagctgcggaggaactttgtgaacattcgcccagccaagttgaagaacctaatcttgctggtgaagcactggtaccaccaggtgtgcctacaggggttgtggaaggagacgctgcccccggtctatgccctggaattgctgaccatcttcgcctgggagcagggctgtaagaaggatgctttcagcctagccgaaggcctccgaactgtcctgggcctgatccaacagcatcagcacctgtgtgttttctggactgtcaactatggcttcgaggaccctgcagttgggcagttcttgcagcggcagcttaagagacccaggcctgtgatcctggacccagctgaccccacatgggacctggggaatggggcagcctggcactgggatttgctagcccaggaggcagcatcctgctatgaccacccatgctttctgagggggatgggggacccagtgcagtcttggaaggggccgggccttccacgtgctggatgctcaggtttgggccaccccatccagctagaccctaaccagaagacccctgaaaacagcaagagcctcaatgctgtgtacccaagagcagggagcaaacctccctcatgcccagctcctggccccactggggcagccagcatcgtcccctctgtgccgggaatggccttggacctgtctcagatccccaccaaggagctggaccgcttcatccaggaccacctgaagccgagcccccagttccaggagcaggtgaaaaaggccattgacatcatcttgcgctgcctccatgagaactgtgttcacaaggcctcaagagtcagtaaagggggctcatttggccggggcacagacctaagggatggctgtgatgttgaactcatcatcttcctcaactgcttcacggactacaaggaccaggggccccgccgcgcagagatccttgatgagatgcgagcgcagctagaatcctggtggcaggaccaggtgcccagcctgagccttcagtttcctgagcagaatgtgcctgaggctctgcagttccagctggtgtccacagccctgaagagctggacggatgttagcctgctgcctgccttcgatgctgtggggcagctcagttctggcaccaaaccaaatccccaggtctactcgaggctcctcaccagtggctgccaggagggcgagcataaggcctgcttcgcagagctgcggaggaacttcatgaacattcgccctgtcaagctgaagaacctgattctgctggtgaagcactggtaccgccaggttgcggctcagaacaaaggaaaaggaccagcccctgcctctctgcccccagcctatgccctggagctcctcaccatctttgcctgggagcagggctgcaggcaggattgtttcaacatggcccaaggcttccggacggtgctggggctcgtgcaacagcatcagcagctctgtgtctactggacggtcaactatagcactgaggacccagccatgagaatgcaccttcttggccagcttcgaaaacccagacccctggtcctggaccccgctgatcccacctggaacgtgggccacggtagctgggagctgttggcccaggaagcagcagcgctggggatgcaggcctgctttctgagtagagacgggacatctgtgcagccctgggatgtgatgccagccctcctttaccaaaccccagctggggaccttgacaagttcatcagtgaatttctccagcccaaccgccagttcctggcccaggtgaacaaggccgttgataccatctgttcatttttgaaggaaaactgcttccggaattctcccatcaaagtgatcaaggtggtcaagggtggctcttcagccaaaggcacagctctgcgaggccgctcagatgccgacctcgtggtgttcctcagctgcttcagccagttcactgagcagggcaacaagcgggccgagatcatctccgagatccgagcccagctggaggcatgtcaacaggagcggcagttcgaggtcaagtttgaagtctccaaatgggagaatccccgcgtgctgagcttctcactgacatcccagacgatgctggaccagagtgtggactttgatgtgctgccagcctttgacgccctaggccagctggtctctggctccaggcccagctctcaagtctacgtcgacctcatccacagctacagcaatgcgggcgagtactccacctgcttcacagagctacaacgggacttcatcatctctcgccctaccaagctgaagagcctgatccggctggtgaagcactggtaccagcagtgtaccaagatctccaaggggagaggctccctacccccacagcacgggctggaactcctgactgtgtatgcctgggagcagggcgggaaggactcccagttcaacatggctgagggcttccgcacggtcctggagctggtcacccagtaccgccagctctgtatctactggaccatcaactacaacgccaaggacaagactgttggagacttcctgaaacagcagcttcagaagcccaggcctatcatcctggatccggctgacccgacaggcaacctgggccacaatgcccgctgggacctgctggccaaggaagctgcagcctgcacatctgccctgtgctgcatgggacggaatggcatccccatccagccatggccagtgaaggctgctgtgtga

SEQ ID NO: 8 - DNA, 297 bp, Homo sapiens
  source 1..297: mol_type = other DNA
atgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaa

SEQ ID NO: 9 - DNA, 1209 bp, Homo sapiens
  source 1..1209: mol_type = other DNA
atgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatgacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagttctccctggagactgaagtcgacctcaggaagcccctagagaacctgggaatgaccgacatgttcagacagtttcaggctgacttcacgagtctttcagaccaagagcctctccacgtcgcgcaggcgctgcagaaagtgaagatcgaggtgaacgagagtggcacggtggcctcctcatccacagctgtcatagtctcagcccgcatggcccccgaggagatcatcatggacagacccttcctctttgtggtccggcacaaccccacaggaacagtccttttcatgggccaagtgatggaaccctga

SEQ ID NO: 10 - DNA, 285 bp, Homo sapiens
  source 1..285: mol_type = other DNA
atgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaa

SEQ ID NO: 11 - DNA, 324 bp, Homo sapiens
  source 1..324: mol_type = other DNA
atggcccacgccacgctctccgccgcccccagcaatccccggctcctgcgggtggcgctgctgctcctgctcctggtggccgccagccggcgcgcagcaggagcgtccgtggtcactgaactgcgctgccagtgcttgcagacactgcagggaattcacctcaagaacatccaaagtgtgaatgtaaggtcccccggaccccactgcgcccaaaccgaagtcatagccacactcaagaatgggaagaaagcttgtctcaaccccgcatcccccatggttcagaaaatcatcgaaaagatactgaacaaggggagcaccaactga